(19) United States
(12) Patent Application Publication
(10) Pub. No.: US 2018/0140280 A1
(43) Pub. Date: May 24, 2018

(54) ULTRASOUND SIGNAL PROCESSING DEVICE, ULTRASOUND DIAGNOSTIC APPARATUS, AND ULTRASOUND SIGNAL PROCESSING METHOD

(71) Applicant: Konica Minolta, Inc., Tokyo (JP)

(72) Inventor: Mineo Tsushima, Kyoto (JP)

(73) Assignee: Konica Minolta, Inc., Tokyo (JP)

(21) Appl. No.: 15/819,600

(22) Filed: Nov. 21, 2017

(30) Foreign Application Priority Data
Nov. 22, 2016 (JP) 2016-227156

(51) Int. Cl. A61B 8/08; A61B 8/14; A61B 8/00; G01S 15/89

(52) U.S. Cl. CPC A61B 8/5207; A61B 8/145; G01S 15/8997; A61B 8/4444; G01S 15/8934; A61B 8/4488

(57) ABSTRACT

An ultrasound signal processing device performs a transmission event, receives reflected ultrasonic waves, generates a received signal sequence, and synthesizes sub-frame acoustic line signals to obtain an acoustic line signal. The device includes: a transmitter that selects a transmitting/receiving oscillator array and directs the transmitting/receiving oscillator array to transmit an ultrasound beam to a target region; a receiver that generates a received signal sequence; a phasing adder that generates a line-region acoustic line signal; an acoustic line signal developer that allocates the line-region acoustic line signal as the acoustic line signal to generate the sub-frame acoustic line signal; and a weighting synthesizer that weights sub-frame acoustic line signals and synthesizes the sub-frame acoustic line signals into a frame acoustic line signal, where, in one of a first weighting profile and a second weighting profile, a first weight sequence has a larger turbulence than a second weight sequence.

Japanese Patent Application No. 2016-227156 filed on Nov. 22, 2016, including description, claims, drawings, and abstract the entire disclosure is incorporated herein by reference in its entirety.

### BACKGROUND

### Technological Field

One or more embodiments of the present disclosure relate to an ultrasound signal processing device and an ultrasound diagnostic apparatus including the same, and more particularly, to a transmission/reception beam forming processing method in an ultrasound signal processing device.

### Description of the Related Art

An ultrasound diagnostic apparatus transmits ultrasonic waves into a subject, using an ultrasound probe (hereinafter, referred to as a “probe”) and receives reflected ultrasonic waves (echoes) generated due to a difference in acoustic impedance between the tissues of the subject. In addition, the ultrasound diagnostic apparatus generates an ultrasound tomographic image indicating the structure of the internal tissues of the subject on the basis of an electric signal obtained from the received reflected ultrasonic waves and displays the ultrasound tomographic image. The ultrasound diagnostic apparatus is minimally invasive to the subject and enables the user to observe the state of the tissues of the body in real time, using, for example, a tomographic image. Therefore, the ultrasound diagnostic apparatus is widely used to diagnose the structure of a living body.

In the related art, a method that is referred to as a phasing addition method has been generally used as a reception beam forming method for a signal based on received reflected ultrasonic waves (for example, Masayasu Itou and Tsuyoshi Mochizuki, “Ultrasound Diagnostic Apparatus”, Corona Publishing Co., Ltd., 26 Aug. 2002, (pp. 42 to 45). In this method, in general, when ultrasonic waves are transmitted to the subject, transmission beam forming is performed such that an ultrasound beam is focused at a certain depth in the subject. In addition, an observation point is set on the central axis of a transmitted ultrasound beam or in the vicinity of the central axis. Therefore, the number of observation points with respect to the area of a main ultrasonic wave irradiation region is small, which results in a low usage efficiency of ultrasonic waves. In addition, in a case in which the observation point is located at a position separated from the vicinity of a transmission focal point, the spatial resolution and S/N ratio of an obtained acoustic line signal are low. The main ultrasonic wave irradiation region indicates a region through which an ultrasound beam is propagated.

In contrast, a reception beam forming method has been proposed which obtains a high-quality image having a high spatial resolution in a region other than the vicinity of a transmission focal point, using a synthetic aperture method (for example, “Virtual ultrasound sources in high resolution ultrasound imaging”, S. I. Nikolov and J. A. Jensen, in Proc, SPIE—Progress in biomedical optics and imaging, vol. 3, 2002, pp. 395-405). According to this method, delay control is performed, considering a propagation path of transmitted ultrasonic waves and the time when reflected waves reach an oscillator through the propagation path, which makes it possible to perform reception beam forming in which ultrasonic waves reflected from the main ultrasonic wave irradiation region other than the vicinity of the transmission focal point are reflected. As a result, it is possible to generate an acoustic line signal for the entire main ultrasonic wave irradiation region in addition to the central axis of the transmitted ultrasound beam (a signal based on the ultrasonic waves reflected from the observation point which is generated by reception beam forming). In addition, in the synthetic aperture method, ultrasonic waves are visually focused on the basis of a plurality of received signals for the same observation point which have been obtained from a plurality of transmission events. Therefore, it is possible to obtain an ultrasound image with a higher spatial resolution and a higher S/N ratio than that in the reception beam forming method described in Masayasu Itou and Tsuyoshi Mochizuki, “Ultrasound Diagnostic Apparatus”, Corona Publishing Co., Ltd., 26 Aug. 2002, (pp. 42 to 45). Patent Literature 1: JP 2008-536578A

In the synthetic aperture method, in order to improve the usage efficiency of ultrasonic waves and resolution, it is possible to increase the area of a region (hereinafter, referred to as a “target region”) in which an acoustic line signal is generated in one ultrasound transmission event. It is also possible to use the main ultrasonic wave irradiation region as the target region. However, the number of observation points (points for which a reception beam forming operation is to be performed) in the target region increases in proportion to the area of the target region. Therefore, when the area of the target region increases, the amount of computation for phasing addition considering delay in transmission and reception increases. For this reason, when the area of the main ultrasonic wave irradiation region increases, hardware with a high operational processing capability is required in order to increase the speed of a phasing addition operation. As a result, the cost of the ultrasound diagnostic apparatus increases. However, when the number of observation points is reduced in order to reduce the amount of computation, the resolution or S/N ratio of an ultrasound image is reduced.

### SUMMARY

One or more embodiments of the invention may provide an ultrasound signal processing device that can significantly reduce the amount of computation while improving spatial resolution and an S/N ratio which is the effect of the synthetic aperture method and an ultrasound diagnostic apparatus using the ultrasound signal processing device.

According to an aspect of one or more embodiments of the present invention, there is provided an ultrasound signal processing device that repeatedly performs a transmission event, which transmits a focused ultrasound beam to a subject using an ultrasound probe including a plurality of oscillators, a plurality of times, receives reflected ultrasonic waves from the subject in synchronization with each transmission event, generates a received signal sequence, and synthesizes a plurality of sub-frame acoustic line signals generated on the basis of the received reflected ultrasonic waves to obtain an acoustic line signal, and the ultrasound signal processing device reflecting one aspect of one or more embodiments of the present invention comprises: a transmitter that selects a transmitting/receiving oscillator array from the plurality of oscillators arranged in a line in the ultrasound probe and directs the transmitting/receiving oscillator array to transmit the ultrasound beam to a target region of the subject for each transmission event, while changing a focal point defining a focus position of the ultrasound beam for each transmission event; a receiver that generates a received signal sequence for each oscillator included in the transmitting/receiving oscillator array on the basis of the reflected ultrasonic waves received from the target region by the ultrasound probe in synchronization with each transmission event; a phasing adder that generates a line-region acoustic line signal from the received signal sequence, using a weighting, phasing, and addition process including a delay process based on a distance between the focal point and each of a plurality of observation points on a straight line passing through the focal point and a distance between the focal point and the oscillator and a weighting process based on a first weighting profile, for each transmission event; an acoustic line signal developer that allocates the line-region acoustic line signal of an observation point, which has the same distance from the focal point as each observation point in the target region and is on the straight line, as the acoustic line signal of the observation point to generate the sub-frame acoustic line signal for each transmission event; and a weighting synthesizer that weights a plurality of sub-frame acoustic line signals related to a plurality of transmission events on the basis of the position of the observation point, using a second weighting profile, and synthesizes the plurality of sub-frame acoustic line signals into a frame acoustic line signal, wherein, in at least one of the first weighting profile and the second weighting profile, a first weight sequence for a first observation point deeper than the focal point has a larger turbulence than a second weight sequence for a second observation point shallower than the focal point.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a functional block diagram illustrating the configuration of an ultrasound diagnostic apparatus according to Embodiment 1;

FIG. 2 is a diagram schematically illustrating a propagation path of transmitted ultrasonic waves by a transmission beam former according to Embodiment 1;

FIG. 3 is a functional block diagram illustrating the configuration of a reception beam former according to Embodiment 1;

FIG. 4 is a functional block diagram illustrating the configuration of a phasing adder according to Embodiment 1;

FIG. 5 is a diagram schematically illustrating a target region, an observation line, an observation point, and a representative point according to Embodiment 1;

FIGS. 6A and 6B are diagrams schematically illustrating the propagation path of ultrasonic waves that travel from a transmitting/receiving aperture to a receiving oscillator through the representative point in Embodiment 1;

FIG. 7 is a diagram schematically illustrating the relationship between the delay times of the receiving oscillators;

FIG. 8 is a diagram schematically illustrating the relationship between the position of the representative point and a weight sequence calculated by a weight calculator in Embodiment 1;

FIG. 9 is a functional block diagram illustrating the configuration of a weighting synthesizer according to Embodiment 1;

FIG. 10 is a diagram schematically illustrating a sub-frame acoustic line signal generation process of an acoustic line signal developer according to Embodiment 1;

FIG. 11 is a diagram schematically illustrating a weighting synthesis process of a weighting synthesizer and a weight sequence in Embodiment 1;

FIGS. 12A and 12B are diagrams schematically illustrating a maximum superimposition number in an acoustic line signal and the outline of an amplification process of a weighting synthesizer in Embodiment 1;

FIG. 13 is a flowchart illustrating a beam forming process of the reception beam former 104 according to Embodiment 1;

FIG. 14 is a flowchart illustrating a line-region acoustic line signal generation operation of the reception beam former according to Embodiment 1;

FIG. 15 is a diagram schematically illustrating an operation of generating an acoustic line signal for the representative point in the reception beam former according to Embodiment 1;

FIG. 16 is a diagram schematically illustrating the relationship between the position of a representative point and a weight sequence calculated by a weight calculator in Modification Example 1;

FIG. 17 is a diagram schematically illustrating a weighting synthesis process of a weighting synthesizer and a weight sequence in Embodiment 2;

FIG. 18 is a diagram schematically illustrating an example of another weight sequence in the weighting synthesizer in Embodiment 2;

FIG. 19 is a diagram schematically illustrating a weight sequence calculated by a weight calculator according to Embodiment 3;

FIG. 20 is a diagram schematically illustrating a weight sequence in a weighting synthesizer according to Embodiment 3;

FIGS. 21A to 21D are diagrams illustrating ultrasound images obtained by reception beam forming according to Comparative Examples 1 to 4; and

FIGS. 22A to 22C are diagrams illustrating ultrasound images obtained by reception beam forming according to Embodiment 1 and Comparative Examples 1 and 3.

### DETAILED DESCRIPTION

Hereinafter, embodiments of the present invention will be described with reference to the drawings. However, the scope of the invention is not limited to the disclosed embodiments.

<<How Embodiments of the Invention Have Been Made>>

The inventors conducted various examinations on an ultrasound diagnostic apparatus using a synthetic aperture method in order to reduce the amount of computation while preventing a reduction in the spatial resolution and S/N ratio of an acoustic line signal (Hereinafter, referred to as “the quality of an acoustic line signal”).

In general, in focusing-type transmission beam forming, a wave front is focused such that an ultrasound beam is focused at a certain depth of a subject (hereinafter, referred to as a “transmission focal depth”). Therefore, a main ultrasonic wave irradiation region is mainly irradiated with ultrasonic waves from a plurality of oscillators (hereinafter, referred to as a “transmitting oscillator array”) used to transmit ultrasonic waves by one ultrasonic wave transmission operation (transmission event). In a case in which there is one transmission focal point, the main ultrasonic wave irradiation region is a sandglass-shaped region that has the transmitting oscillator array as the base and is surrounded by two straight lines that extend from both ends of the base and pass through the transmission focal point and a wave front has an arc shape having the transmission focal point as the center. In addition, the ultrasound beam is not necessarily focused on one point. For example, in some cases, the ultrasound beam is focused on a region with a size corresponding to one and a half oscillators to several oscillators. In this case, the main ultrasonic wave irradiation region has a shape in which the width in the array direction is reduced toward the transmission focal depth, is equal to the width of a focus region in the array direction at the transmission focal depth, and increases in the array direction in a region of which the depth is greater than the transmission focal depth. In this case, the center point of the focus region at the transmission focal depth is defined as a “transmission focal point” for convenience. That is, the main ultrasonic wave irradiation region has a shape which is converged on the transmission focal point or in the vicinity of the transmission focal point at the transmission focal depth and the width in the array direction (a direction in which elements are arranged) increases as the distance to the transmission focal depth increases, regardless of whether there is one focal point.

In the synthetic aperture method, the observation points can be set in the entire main ultrasonic wave irradiation region in one transmission event. Therefore, it is possible to use the entire main ultrasonic wave irradiation region as a target region. In one transmission event, it is difficult to set the entire region for generating an ultrasound image (hereinafter, referred to as “a region of interest”) as the target region. Therefore, a plurality of transmission events with different target regions are performed in order to generate one frame of ultrasound images. It is possible that the area of the target region in the main ultrasonic wave irradiation region be large in one transmission event in order to improve the usage efficiency of ultrasonic waves. In general, it is possible that the overlap area between the target regions in two consecutive transmission events be large in order to improve spatial resolution or the S/N ratio of a signal under the static condition in which the target region is not moved.

However, the number of observation points in the target region, that is, the number of points for which a reception beam forming operation is to be performed is proportional to the area of the target region. Therefore, the amount of computation for phasing addition and a memory size required to store the acoustic line signal after phasing addition are proportional to the area of the target region. An increase in the area of the target region leads to an increase in the amount of computation and the memory size of the ultrasound diagnostic apparatus. In addition, when the computing capability of the ultrasound diagnostic apparatus with respect to the amount of computation for phasing addition is insufficient, it is difficult for the frame rate to be greater than a value suitable for the computing capability. The frame rate of the ultrasound images is reduced, which results in a reduction in temporal resolution. As a result, usability is reduced. Therefore, a processor with high processing capability to perform a phasing addition operation at a high speed, for example, a high-performance GPU is required in order to prevent the reduction in temporal resolution or the reduction in usability, which causes an increase in the cost of the ultrasound diagnostic apparatus.

A method that reduces the number of observation points included in the target region is considered in order to reduce the amount of computation. However, when the number of observation points is reduced more than necessary to reduce the amount of computation, the resolution or S/N ratio of an ultrasound image is reduced in operative association with the amount of computation. In addition, in some cases, only the reduction in the number of observation points is not radically enough to reduce the amount of computation. For this reason, the inventors conducted an examination on a method for performing phasing addition at only some observation points and generating acoustic line signals for the entire region of interest, using the results of the phasing addition, in order to reduce the amount of computation while obtaining the advantages of the synthetic aperture method. According to this method, for example, it is possible to radically reduce the amount of computation for phasing addition, as disclosed in “Synthetic Aperture Sequential Beam forming”, Jacob Kortbek, et al, IEEE Ultrasonics Symposium, 2-5 Nov. 2008, pp. 966-969. In addition, the inventors conducted a research on a method for optimizing the balance of the quality of an acoustic line signal and found the idea that a weight sequence for a receiving element array was changed in phasing addition and/or a weight sequence for an acoustic line signal was changed in the synthesis of acoustic lines, according to whether the depth of the observation point was greater than the transmission focal depth. Specifically, in a case in which the depth of the observation point is greater than the transmission focal depth, a weight sequence in which there is a large change in a weight is used. In a case in which the depth of the observation point is less than the transmission focal depth, a flat weight sequence is used. In this way, in a region of which the depth is less than the transmission focal depth, it is possible to prevent a reduction in the S/N ratio and thus to prevent the user from feeling that the quality of an ultrasound image is degraded due to the roughness of a spectrum. In a region of which the depth is greater than the transmission focal depth, it is possible to ensure a high spatial resolution.

Hereinafter, an ultrasound image processing method and an ultrasound diagnostic apparatus using the same according to embodiments will be described in detail below with reference to the drawings.

### Embodiment 1

<Overall Configuration>

Hereinafter, an ultrasound diagnostic apparatus 100 according to Embodiment 1 will be described with reference to the drawings.

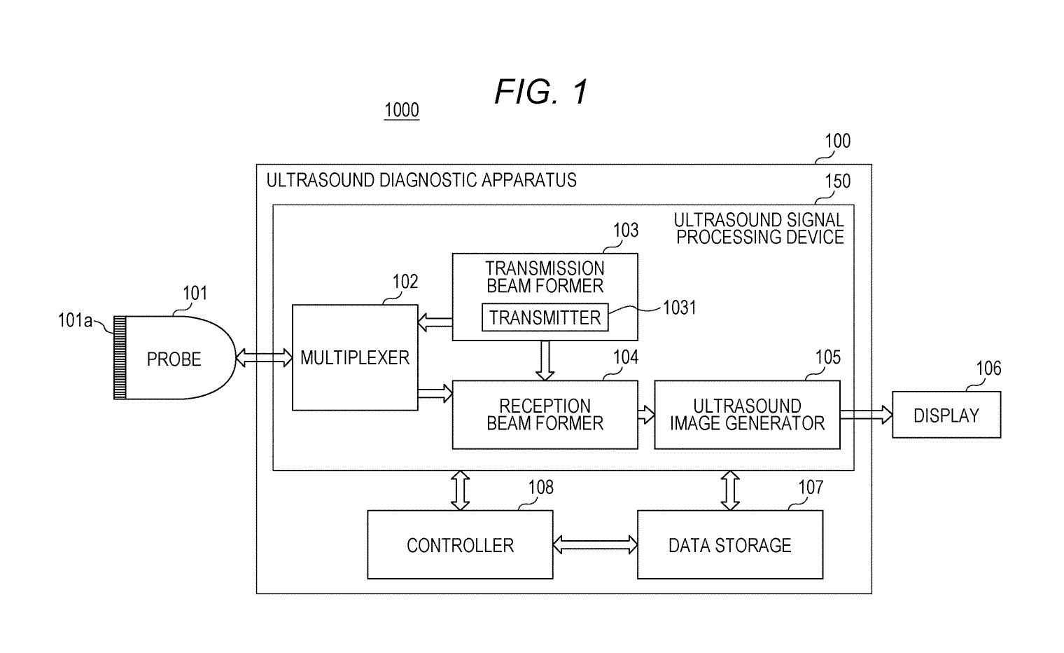

FIG. 1 is a functional block diagram illustrating an ultrasound diagnostic system 1000 according to the embodiment. As illustrated in FIG. 1, the ultrasound diagnostic system 1000 includes a probe 101 including a plurality of oscillators 101a that transmit ultrasonic waves to a subject and receive reflected waves, the ultrasound diagnostic apparatus 100 that directs the probe 101 to transmit and receive ultrasonic waves and generates an ultrasound image on the basis of an output signal from the probe 101, and a display 106 that displays the ultrasound image on a screen. Each of the probe 101 and the display 106 is configured so as to be connected to the ultrasound diagnostic apparatus 100. FIG. 1 illustrates a state in which the probe 101 and the display 106 are connected to the ultrasound diagnostic apparatus 100. In addition, the probe 101 and the display 106 may be provided in the ultrasound diagnostic apparatus 100.

The ultrasound diagnostic apparatus 100 includes a multiplexer 102 that selects each oscillator used for transmission or reception among the plurality of oscillators 101a of the probe 101 and ensures an input and an output to and from the selected oscillator, a transmission beam former 103 that controls the time when a high voltage is applied to each oscillator 101a of the probe 101 in order to transmit ultrasonic waves, and a reception beam former 104 that amplifies an electric signal obtained by the plurality of oscillators 101a on the basis of the reflected waves of the ultrasonic waves received by the probe 101, converts an analog electric signal into a digital electric signal, and performs reception beam forming to generate an acoustic line signal. In addition, the ultrasound diagnostic apparatus 100 includes an ultrasound image generator 105 that generates an ultrasound image (B-mode image) on the basis of the signal output from the reception beam former 104, a data storage 107 that stores the acoustic line signal output from the reception beam former 104 and the ultrasound image output from the ultrasound image generator 105, and a controller 108 that controls each component.

Among them, the multiplexer 102, the transmission beam former 103, the reception beam former 104, and the ultrasound image generator 105 form an ultrasound signal processing device 150.

Each of the elements forming the ultrasound diagnostic apparatus 100, for example, the multiplexer 102, the transmission beam former 103, the reception beam former 104, and the ultrasound image generator 105, and the controller 108 is implemented by a hardware circuit, such as a field programmable gate array (FPGA) or an application specific integrated circuit (ASIC). Alternatively, each element may be implemented by a programmable device, such as a processor, and software. A central processing unit (CPU) or a general-purpose computing on graphics processing unit (GPGPU) can be used as the processor. A configuration using a GPU is referred to as a GPGPU. Each of the components may be a single circuit component or may be an aggregate of a plurality of circuit components. In addition, a plurality of components may be integrated into one circuit component or an aggregate of a plurality of circuit components may be used.

The data storage 107 is a computer-readable recording medium. For example, a flexible disk, a hard disk, a MO, a DVD, a DVD-RAM, a BD, or a semiconductor memory can be used as the data storage 107. In addition, the data storage 107 may be an external storage device connected to the ultrasound diagnostic apparatus 100.

The ultrasound diagnostic apparatus 100 according to this embodiment is not limited to the ultrasound diagnostic apparatus having the configuration illustrated in FIG. 1. For example, the multiplexer 102 may not be provided and the transmission beam former 103 and the reception beam former 104 may be directly connected to each oscillator 101a of the probe 101. In addition, for example, the transmission beam former 103 or the reception beam former 104 may be provided in the probe 101 or a portion thereof may be provided in the probe 101. This configuration is not limited to the ultrasound diagnostic apparatus 100 according to this embodiment and holds for ultrasound diagnostic apparatuses according to other embodiments or modification examples which will be described below.

<Configuration of Main Portion of Ultrasound Diagnostic Apparatus 100>

The ultrasound diagnostic apparatus 100 according to Embodiment 1 is characterized by the transmission beam former 103 that directs each oscillator 101a of the probe 101 to transmit ultrasonic waves and the reception beam former 104 that processes the electric signal obtained from the reflected waves of the ultrasonic waves received by the probe 101 to generate an acoustic line signal for generating an ultrasound image. Therefore, in the specification, the configuration and function of the transmission beam former 103 and the reception beam former 104 will be mainly described. In addition, the same configuration as that used in a known ultrasound diagnostic apparatus can be applied to components other than the transmission beam former 103 and the reception beam former 104 and the beam former according to this embodiment can be replaced with the beam former of the known ultrasound diagnostic apparatus.

Next, the configuration of the transmission beam former 103 and the reception beam former 104 will be described below.

1. Transmission Beam Former 103

The transmission beam former 103 is connected to the probe 101 through the multiplexer 102 and controls the time when a high voltage is applied to each of a plurality of oscillators included in a transmitting/receiving aperture TRx formed by a transmitting oscillator array that corresponds to some or all of the plurality of oscillators 101a in the probe 101 in order to transmit ultrasonic waves from the probe 101. The transmission beam former 103 includes a transmitter 1031.

The transmitter 1031 is a circuit that performs a transmission process of supplying a pulsed transmission signal for directing each oscillator included in the transmitting/receiving aperture TRx among the plurality of oscillators 101a in the probe 101 to transmit an ultrasound beam, on the basis of a transmission control signal from the controller 108. Specifically, the transmitter 1031 includes, for example, a clock generation circuit, a pulse generation circuit, and a delay circuit. The clock generation circuit generates a clock signal for determining the time when an ultrasound beam is transmitted. The pulse generation circuit generates a pulse signal for driving each oscillator. The delay circuit sets a delay time to the time when each oscillator transmits an ultrasound beam, delays the transmission of the ultrasound beam by the delay time, and focuses the ultrasound beam.

The transmitter 1031 repeatedly transmits ultrasonic waves while moving the transmitting/receiving aperture TRx by a transmission pitch Mp with a fixed length in an array direction whenever the ultrasonic waves are transmitted such that all of the oscillators 101a in the probe 101 transmit ultrasonic waves. In this embodiment, the transmission pitch Mp is a distance corresponding to one oscillator. That is, in this embodiment, whenever ultrasonic waves are transmitted, the transmitting/receiving aperture TRx is moved by a distance corresponding to one oscillator. The transmission pitch Mp is not limited to the above-mentioned example. For example, the transmission pitch Mp is a distance corresponding to half of the oscillator. Information indicating the position of the oscillator included in the transmitting/receiving aperture TRx is output to the data storage 107 through the controller 108. For example, when the total number of oscillators 101a in the probe 101 is 192, 20 to 100 oscillator arrays may be selected as the oscillator arrays forming the transmitting/receiving aperture TRx and the transmission pitch Mp may be a distance corresponding to one oscillator. Hereinafter, the transmission of ultrasonic waves from the same transmitting/receiving aperture TRx by the transmitter 1031 is referred to as a “transmission event”.

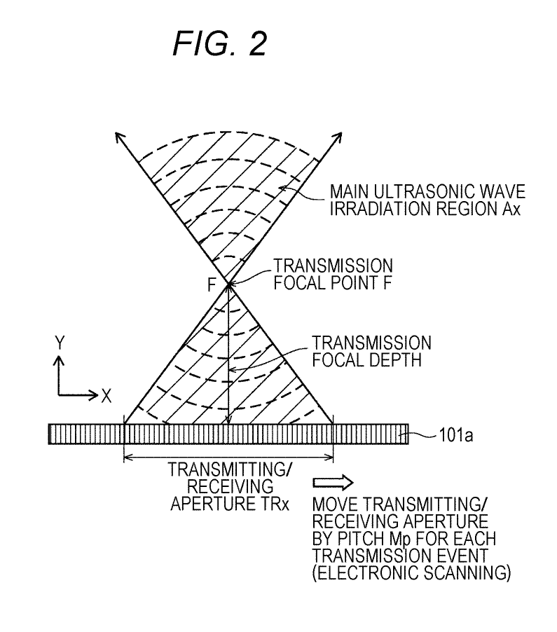

FIG. 2 is a diagram schematically illustrating a propagation path of the ultrasonic waves transmitted by the transmission beam former 103. In a certain transmission event, a row of the oscillators 101a (transmitting oscillator array) which contribute to the transmission of ultrasonic waves and are arranged in an array is illustrated as the transmitting/receiving aperture TRx. In addition, the transmitting/receiving aperture TRx is referred to as a transmitting/receiving aperture length.

The transmission beam former 103 controls the transmission time of each oscillator such that the oscillator closer to the center of the transmitting/receiving aperture TRx has a longer transmission delay time. In this case, the ultrasonic waves transmitted from the oscillator array in the transmitting/receiving aperture TRx are focused at a point with a wave front, that is, a transmission focal point F at a certain focal depth of the subject. The depth (focal depth) at the transmission focal point F can be arbitrarily set. The wave front focused at the transmission focal point F is diffused again. The transmitted ultrasonic waves are propagated through a sandglass-shaped space that is demarcated by two straight lines crossing each other, has the transmitting/receiving aperture TRx as the base, and has the transmission focal point F as a node. That is, the width (in the horizontal direction in FIG. 2) of the ultrasonic waves radiated from the transmitting/receiving aperture TRx is gradually reduced in the space and is minimized at the transmission focal point F. Then, as the ultrasonic waves travel to a deeper portion (an upper portion in FIG. 2), the ultrasonic waves are more diffused while the width of the ultrasonic waves becomes larger. Then, the ultrasonic waves are propagated. The sandglass-shaped region is a main ultrasonic wave irradiation region Ax. As described above, ultrasonic waves may be transmitted such that the main ultrasonic wave irradiation region Ax is converged in the vicinity of one transmission focal point F.

2. Configuration of Reception Beam Former 104

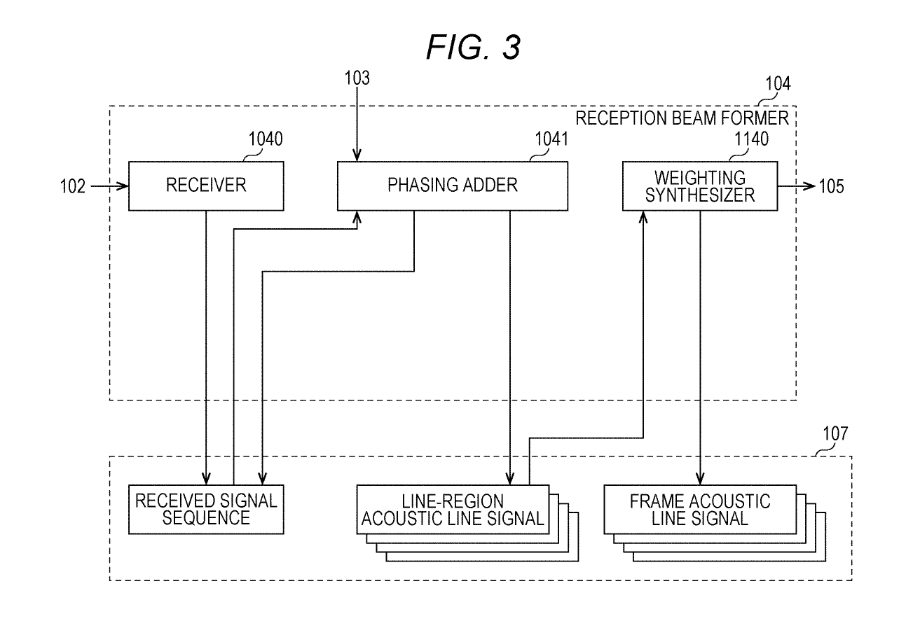

The reception beam former 104 generates an acoustic line signal from the electric signal obtained by the plurality of oscillator 101a, on the basis of the reflected waves of the ultrasonic waves received by the probe 101. The “acoustic line signal” is a signal obtained by performing a phasing addition process for a certain observation point. The phasing addition process will be described below. FIG. 3 is a functional block diagram illustrating the configuration of the reception beam former 104. As illustrated in FIG. 3, the reception beam former 104 includes a receiver 1040, a phasing adder 1041, and a weighting synthesizer 1140.

Next, the configuration of each component forming the reception beam former 104 will be described below.

(1) Receiver 1040

The receiver 1040 is a circuit that is connected to the probe 101 through the multiplexer 102, amplifies an electric signal obtained from the reflected waves of the ultrasonic waves received by the probe 101 in synchronization with a transmission event and performs A/D conversion for the electric signal to generate a received signal (RF signal). The receiver 1040 generates the received signal in time series in the order of the transmission events, outputs the received signal to the data storage 107, and stores the received signal in the data storage 107.

Here, the received signal (RF signal) is a digital signal obtained by converting the reflected ultrasonic waves received by each oscillator into an electric signal and performing A/D conversion for the electric signal and forms a sequence of signals arranged in the transmission direction (the depth direction of the subject) of the ultrasonic waves received by each oscillator.

In a transmission event, as described above, the transmitter 1031 directs each of a plurality of oscillators included in the transmitting/receiving aperture TRx among the plurality of oscillators 101a in the probe 101 to transmit an ultrasound beam. In contrast, the receiver 1040 generates a sequence of the received signals for each oscillator on the basis of the reflected ultrasonic waves obtained by each of the plurality of oscillators included in the transmitting/receiving aperture TRx in synchronization with the transmission event. Here, the oscillator that receives the reflected ultrasonic waves is referred to as a “wave receiving oscillator”. In this embodiment, all of the oscillators included in the transmitting oscillator array are used as the wave receiving oscillators. Therefore, the number of wave receiving oscillators is equal to the number of oscillators included in a transmitting aperture Tx.

The transmitter 1031 repeatedly transmits ultrasonic waves while moving the transmitting/receiving aperture TRx by the transmission pitch MP in the array direction to transmit ultrasonic waves from all of the plurality of oscillators 101a in the probe 101 in synchronization with the transmission event. The receiver 1040 generates a sequence of the received signals for each wave receiving oscillator in synchronization with the transmission event. The generated received signal is stored in the data storage 107.

(2) Phasing Adder 1041

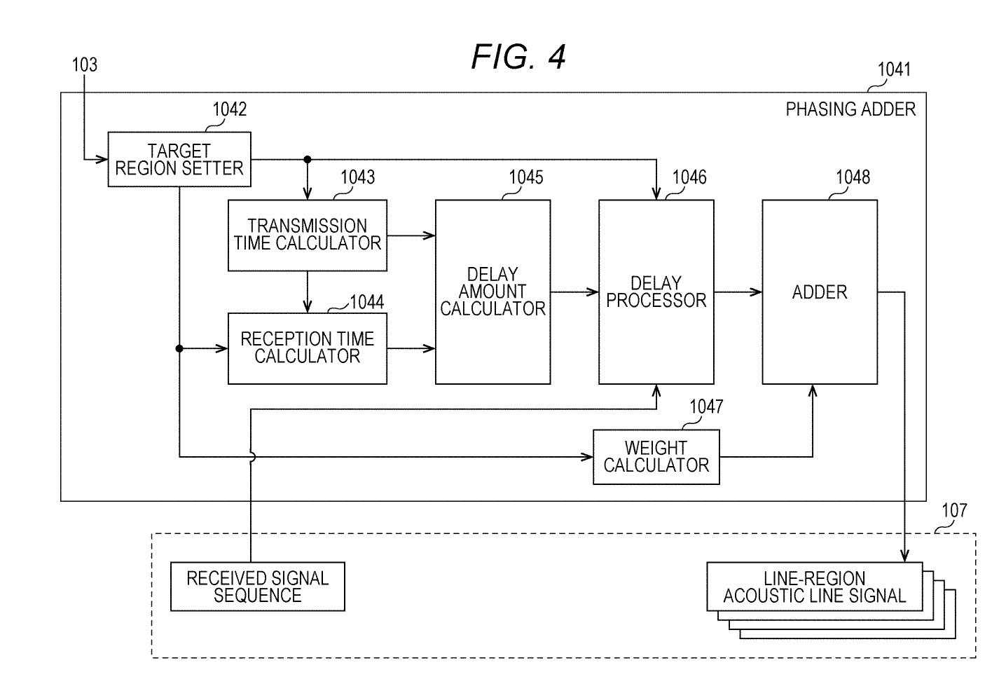

The phasing adder 1041 is a circuit that generates a line-region acoustic line signal from the received signal sequence. First, the phasing adder 1041 sets a target region Bx in which a sub-frame acoustic line signal is generated in the subject in synchronization with the transmission event. Then, the phasing adder 1041 sets an observation line BL that passes through the transmission focal point F in the target region Bx. In this embodiment, the observation line BL is a straight line that passes through the transmission focal point F and the center line of the transmitting/receiving aperture TRx and perpendicular to an oscillator array. In addition, the observation line BL may pass through the transmission focal point F and an arbitrary point in the transmitting/receiving aperture TRx and is not limited to the above-mentioned case. Then, the phasing adder 1041 performs phasing addition for the received signal sequence, which has been received from each of a plurality of representative points Qk on the observation line BL by each receiving oscillator Rm, for each representative point Qk. Then, the phasing adder 1041 calculates an acoustic line signal sequence at each representative point Qk to generate a line-region acoustic line signal. FIG. 4 is a functional block diagram illustrating the configuration of the phasing adder 1041. As illustrated in FIG. 4, the phasing adder 1041 includes a target region setter 1042, a transmission time calculator 1043, a reception time calculator 1044, a delay amount calculator 1045, a delay processor 1046, a weight calculator 1047, and an adder 1048.

Next, the configuration of each component forming the phasing adder 1041 will be described below.

i) Target Region Setter 1042

The target region setter 1042 sets the target region Bx in which the sub-frame acoustic line signal is generated in the subject. The “target region” is a region on a signal in which the sub-frame acoustic line signal is to be generated in the subject in synchronization with the transmission event. An acoustic line signal is generated for an observation point Pij in the target region Bx. The target region Bx is a set of observation target points where the acoustic line signal is generated and is set in synchronization with one transmission event for convenience of calculation.

Here, the “sub-frame acoustic line signal” is a set of acoustic line signals for all of the observation points Pij in the target region Bx generated from one transmission event. The “sub-frame” is obtained in one transmission event and is the unit of the formation of a set of signals corresponding to all of the observation points Pij in the target region Bx. A plurality of sub-frames acquired at different times are synthesized into a frame.

The target region setter 1042 sets the target region Bx on the basis of information indicating the position of the transmitting/receiving aperture TRx acquired from the transmission beam former 103, in synchronization with the transmission event.

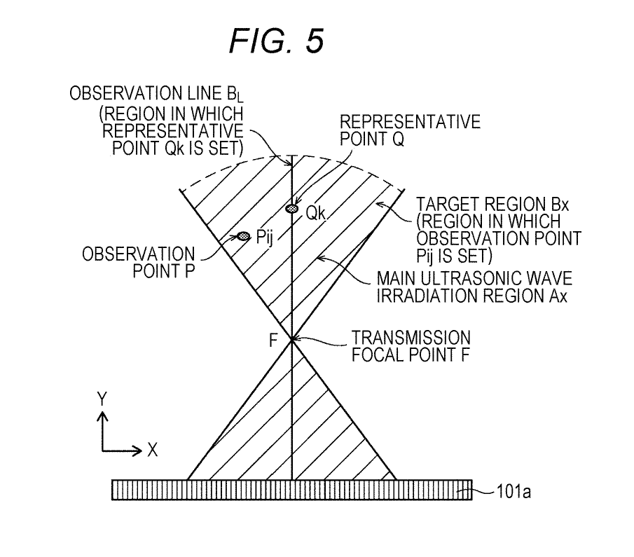

FIG. 5 is a diagram schematically illustrating the target region Bx. As illustrated in FIG. 5, the target region Bx is an arbitrary region in the main ultrasonic wave irradiation region Ax. In this embodiment, the target region Bx is the entire main ultrasonic wave irradiation region Ax.

In addition, the target region setter 1042 sets a target line BL where the line-region acoustic line signal is generated in the target region Bx. The target line BL is a straight line that passes through the focal point F or the vicinity of the focal point F. In this embodiment, the target line BL passes through the center of the transmitting/receiving aperture TRx. However, the target line BL may be a region on a straight line that passes through the focal point F or the vicinity of the focal point F and an arbitrary point on the transmitting/receiving aperture TRx. Then, the line-region acoustic line signal is generated for the representative point Qk on the target line BL.

The set target region Bx, the set target line BL, and the transmitting/receiving aperture TRx acquired from the transmission beam former 103 are output to the transmission time calculator 1043, the reception time calculator 1044, the delay processor 1046, and the weight calculator 1047.

ii) Transmission Time Calculator 1043

The transmission time calculator 1043 is a circuit that calculates the transmission time when the transmitted ultrasonic waves reach an observation point P in the subject. The transmission time calculator 1043 calculates the transmission time when the transmitted ultrasonic waves reach an arbitrary representative point Qk on the target line BL in the subject on the basis of information indicating the position of the oscillators included in the transmitting/receiving aperture TRx and information indicating the position of the target line BL which have been acquired from the target region setter 1042, in correspondence with the transmission event.

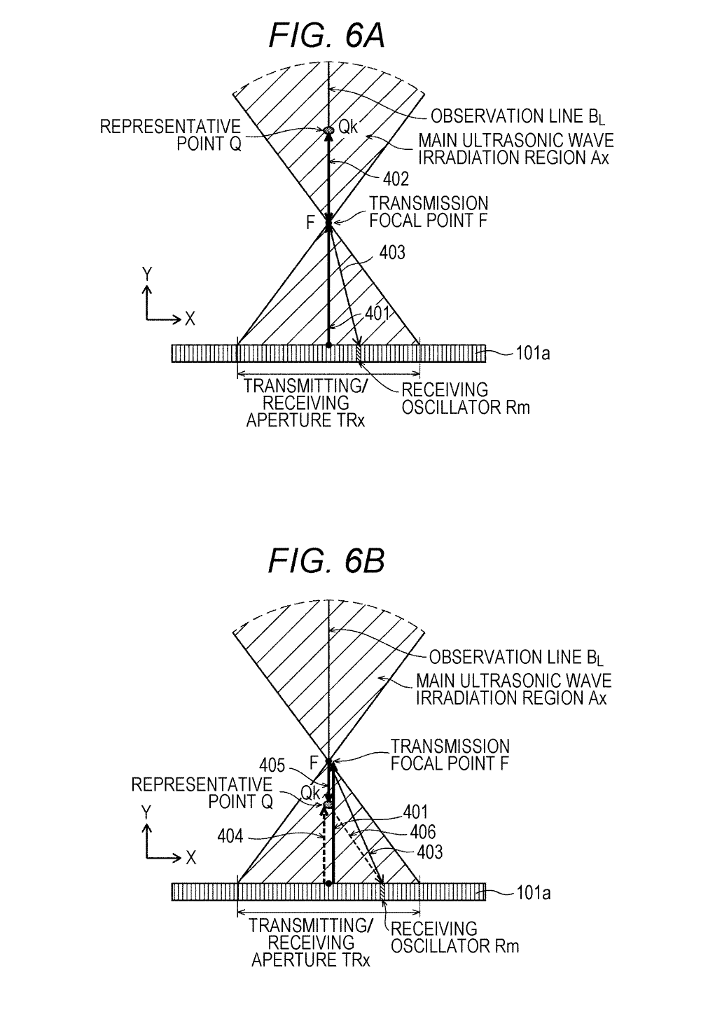

FIGS. 6A and 6B are diagrams schematically illustrating the propagation path of the ultrasonic waves which are radiated from the transmitting/receiving aperture TRx, are reflected from a representative point Qk at an arbitrary position on the target line BL, and reach the receiving oscillator Rm in the transmitting/receiving aperture TRx. FIG. 6A illustrates a case in which the depth of the representative point Qk is equal to or greater than the transmission focal depth and FIG. 6B illustrates a case in which the depth of the representative point Qk is less than the transmission focal depth.

The wave fronts of the transmitted waves radiated from the transmitting/receiving aperture TRx are focused on the transmission focal point F through a path 401 and are diffused again. The transmitted waves reach the representative point Qk while being focused or diffused. When acoustic impedance changes at the representative point Qk, reflected waves are generated. The reflected waves return to the receiving oscillator Rm in the transmitting/receiving aperture TRx in the probe 101. The transmission focal point F is defined as the design value of the transmission beam former 103. Therefore, it is possible to geometrically calculate the length of a path 402 between the transmission focal point F and an arbitrary representative point Qk.

Next, a transmission time calculation method will be described in detail below.

First, a case in which the depth of the representative point Qk is equal to or greater than the transmission focal depth will be described below with reference to FIG. 6A. In a case in which the depth of the representative point Qk is equal to or greater than the transmission focal depth, the transmitted waves radiated from the transmitting/receiving aperture TRx reach the transmission focal point F through the path 401 and then travel from the transmission focal point F to the representative point Qk through the path 402. The transmission time is calculated using the transmitted waves. Therefore, the sum of the time when the transmitted waves pass through the path 401 and the time when the transmitted waves pass through the path 402 is the transmission time. As a specific calculation method, for example, the total length of the paths, which is the sum of the length of the path 401 and the length of the path 402, is divided by the propagation velocity of ultrasonic waves in the subject to calculate the transmission time.

In contrast, a case in which the depth of the representative point Qk is less than the transmission focal depth will be described below with reference to FIG. 6B. In a case in which the depth of the representative point Qk is less than the transmission focal depth, the time when the transmitted waves radiated from the transmitting/receiving aperture TRx reach the transmission focal point F through the path 401 and the time when the transmitted waves reach the representative point Qk through the path 404 and then travel from the representative point Qk to the transmission focal point F through a path 405 are calculated to be equal to each other. That is, a value obtained by subtracting the time when the transmitted waves pass through the path 405 from the time when the transmitted waves pass through the path 401 is the transmission time. As a specific calculation method, for example, a path length difference obtained by subtracting the length of the path 405 from the length of the path 401 is divided by the propagation velocity of ultrasonic waves in the subject to calculate the transmission time.

The transmission time in a case in which the depth of the representative point Qk is equal to the transmission focal depth is calculated by the same calculation method as that in a case in which the depth of the representative point Qk is greater than the transmission focal depth, that is, the calculation method that adds the time when the transmitted waves pass through the path 401 and the time when the transmitted waves pass through the path 402. However, the transmission time may be calculated by the calculation method as that in a case in which the depth of the representative point Qk is less than the transmission focal depth, that is, the calculation method that subtracts the time when the transmitted waves pass through the path 405 from the time when the transmitted waves pass through the path 401. The reason is that, since both the length of the path 402 and the length of the path 405 are 0, the transmission time is equal to the time when the transmitted waves pass through the path 401 even when it is calculated at any point.

The transmission time calculator 1043 calculates the transmission time when the transmitted ultrasonic waves reach the observation point Qk in the subject for all of the representative points Qk on the target line BL in one transmission event and outputs the transmission time to the delay amount calculator 1045. In addition, the transmission time calculator 1043 outputs the length of the path 402 or the path 405 for all of the representative points Qk on the target line BL in one transmission event to the reception time calculator 1044.

iii) Reception Time Calculator 1044

The reception time calculator 1044 is a circuit that calculates the reception time when the waves reflected from the representative point Q reach each of the receiving oscillators Rm included in the transmitting/receiving aperture TRx. The reception time calculator 1044 calculates the reception time when the transmitted ultrasonic waves are reflected from an arbitrary representative point Qk on the target line BL in the subject and reach each receiving oscillators Rm in the transmitting/receiving aperture TRx on the basis of information indicating the position of a receiving oscillator Rk and the information indicating the position of the target line BL which have been acquired from the target region setter 1042, in correspondence with the transmission event.

As described above, when there is a change in acoustic impedance at the representative point Qk, the reflected waves of the transmitted waves are generated at the representative point Qk. The reflected waves return to each receiving oscillator Rm included in the transmitting/receiving aperture TRx in the probe 101. In this case, the reception time calculator 1044 calculates a path from the representative point Qk to the receiving oscillator Rm on the basis of the transmission focal point F, similarly to the transmission ultrasound beam.

First, the concept of a reception time calculation method will be described with reference to FIGS. 6A and 6B. However, calculation can be simplified as described below.

First, a case in which the depth of the representative point Qk is equal to or greater than the transmission focal depth will be described with reference to FIG. 6A. In a case in which the depth of the representative point Qk is equal to or greater than the transmission focal depth, the waves reflected from the representative point Qk reach the transmission focal point F through the path 402 and travel from the transmission focal point F to the receiving oscillator Rm through the path 403. The reception time is calculated using the reflected waves. Therefore, the sum of the time when the reflected waves pass through the path 402 and the time when the reflected waves pass through the path 403 is the reception time.

In contrast, a case in which the depth of the representative point Qk is less than the transmission focal depth will be described with reference to FIG. 6B. In a case in which the depth of the representative point Qk is less than the transmission focal depth, the time when the waves reflected from the transmission focal point F reach the representative point Qk through the path 405 and reach the receiving oscillator Rm through a path 406 is calculated to be equal to the time when the reflected waves directly reach the receiving oscillator Rm through a path 403. In other words, the time when the waves reflected from the representative point Qk reach the receiving oscillator Rm is shorter than the time when the waves reflected from the transmission focal point F reach the receiving oscillator Rm through the path 403 by the time required for the reflected waves to pass through the path 405. Therefore, a value obtained by subtracting the time when the reflected waves pass through the path 405 from the time when the reflected waves pass through the path 403 is the reception time.

Here, the length of the path 402 or the path 405 for each representative point Qk is equal to the length of the path 402 or the path 405 for each representative point Qk which is calculated as a portion of the transmission time by the transmission time calculator 1043. Therefore, in this embodiment, the length of the path 402 or the path 405 for each representative point Qk which is calculated by the transmission time calculator 1043 is acquired and used to calculate the reception time. In addition, the length of the path 403 depends on only the positional relationship between the transmission focal point F and the receiving oscillator Rm. In other words, the difference in reception time between two receiving oscillators Rm₁ and Rm₂ is the same for a representative point Qk₁, a representative point Qk₂, and a representative point Qk₃.

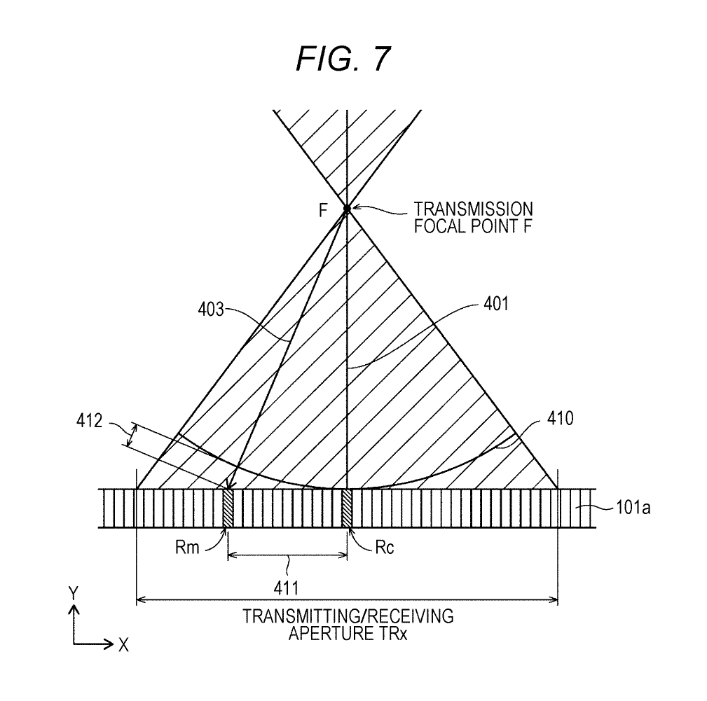

Next, this will be described in detail with reference to FIG. 7. The length of the path 403 is determined by the positional relationship between the receiving oscillator Rm and the transmission focal point F. The difference between the reception time of the receiving oscillator Rm and the reception time of a receiving oscillator Rc located at the center of the transmitting/receiving aperture TRx is the time required for ultrasonic waves to travel a distance 412 between the receiving oscillator Rm and an arc 410 that has the transmission focal point F as the center and is tangent to the receiving oscillator Rc.

Therefore, the reception time calculator 1044 calculates the reception time of the receiving oscillator Rc for each representative point Qk, using the length of the path 401 corresponding to the length of the path 403 of the receiving oscillator Rc and the length of the path 402 or the path 405 for each representative point Qk calculated by the transmission time calculator 1043. In addition, the distance 412 for each receiving oscillator Rm is divided by the propagation velocity of the ultrasonic waves to calculate the difference between the reception time of each receiving oscillator Rm and the reception time of the receiving oscillator Rc. Then, the reception time calculator 1044 outputs the reception time of the receiving oscillator Rc for each representative point Qk and the difference between the reception time of each receiving oscillator Rm and the reception time of the receiving oscillator Rc to the delay amount calculator 1045.

iv) Delay Amount Calculator 1045

The delay amount calculator 1045 is a circuit that calculates the total propagation time of each receiving oscillator Rm in the transmitting/receiving aperture TRx from the transmission time and the reception time and calculates the amount of delay applied to the received signal sequence for each receiving oscillator Rm on the basis of the total propagation time. The delay amount calculator 1045 acquires the transmission time when the transmitted ultrasonic waves reach the representative point Qk, the reception time when the ultrasonic waves are reflected from the representative point Qk and reach the receiving oscillator Rc, and the difference between the reception time of the receiving oscillator Rc and the reception time of each receiving oscillator Rm. Then, the delay amount calculator 1045 calculates the total propagation time until the transmitted ultrasonic waves reach each receiving oscillator Rm, and calculates the amount of delay for each receiving oscillator Rm on the basis of a difference in the total propagation time between the receiving oscillators Rm. The total propagation time of the receiving oscillator Rc for each representative point Qk can be obtained as the sum of the transmission time for the representative point Qk and the reception time of the receiving oscillator Rc. In addition, the total propagation time of each receiving oscillator Rm can be obtained by adding the difference between the reception time of the receiving oscillator Rc and the reception time of each receiving oscillator Rm to the total propagation time of the receiving oscillator Rc for the same representative point Qk. The delay amount calculator 1045 calculates the amount of delay to be applied to the received signal sequence for the receiving oscillator Rc for all of the representative points Qk on the target line BL and outputs both the amount of delay and the difference between the reception time of the receiving oscillator Rc and the reception time of each receiving oscillator Rm to the delay processor 1046.

v) Delay Processor 1046

The delay processor 1046 is a circuit that identifies a received signal corresponding to the amount of delay for each receiving oscillator Rm as a received signal corresponding to each receiving oscillator Rm based on the ultrasonic waves reflected from the representative point Qk from the received signal sequence for the receiving oscillator Rm in the transmitting/receiving aperture TRx.

The delay processor 1046 acquires, as inputs, the information indicating the position of the receiving oscillator Rm and the position of the target line BL which has been output from the target region setter 1042, the received signal corresponding to the receiving oscillator Rm which has been output from the data storage 107, and the amount of delay applied to the received signal sequence for each receiving oscillator Rm which has been output from the delay amount calculator 1045, in correspondence with the transmission event. Then, the delay processor 1046 identifies a received signal corresponding to the time obtained by subtracting the amount of delay for each receiving oscillator Rm from the received signal sequence corresponding to each receiving oscillator Rm as a received signal based on the waves reflected from the representative point Qk and outputs the received signal to the adder 1048.

Specifically, the delay processor 1046 performs a delay process for the received signal sequence for each receiving oscillator Rm such that the difference between the reception time of the receiving oscillator Rc and the reception time of each receiving oscillator Rm is removed. The delay processor 1046 extracts the received signals corresponding to the same time from the received signal sequence subjected to the delay process to extract a set of the received signals based on the ultrasonic waves reflected from the same representative point Qk.

vi) Weight Calculator 1047

The weight calculator 1047 is a circuit that calculates a weight sequence (receiving apodization) for each receiving oscillator Rm.

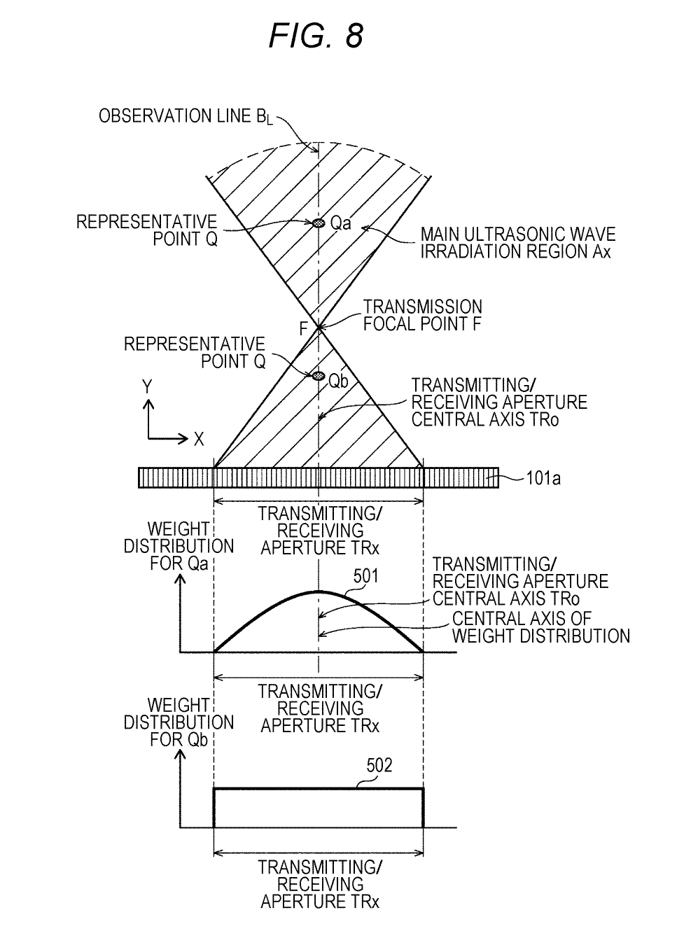

As illustrated in FIG. 8, the weight sequence is a sequence of weighting coefficients applied to the received signals corresponding to each oscillator in the transmitting/receiving aperture TRx. The weight sequence has a symmetric distribution with respect to the center of the transmission focal point F. The weight sequence is set such that a weight for the oscillator located at the center of the transmitting/receiving aperture TRx in the array direction is the maximum. The central axis of a weight distribution is aligned with a transmitting/receiving aperture central axis TRo.

The weight sequence is different in a case in which the depth of the representative point Q is equal to or greater than the transmission focal depth and a case in which the depth of the representative point Q is less than the transmission focal depth. Specifically, a weight sequence 501 of which the distribution shape is a Hamming window is used for a representative point Qa of which the depth is equal to or greater than the transmission focal depth. In contrast, a weight sequence 502 of which the distribution shape is a rectangular window is used for a representative point Qb of which the depth is less than the transmission focal depth.

The reason is as follows. In a case in which the depth of the representative point is equal to or greater than the transmission focal depth, it is possible to improve spatial resolution. Therefore, the weight sequence is set such that a weight for the receiving oscillator closest to the representative point Q, that is, the oscillator located at the center of the transmitting/receiving aperture TRx in the array direction is the maximum and a smaller weight is applied to the receiving oscillator which becomes further away from the representative point Q, that is, a smaller weight is applied to the receiving oscillator closer to the end of the transmitting/receiving aperture TRx. It is possible that each weight in the weight sequence be reduced depending on the distance from the transmitting/receiving aperture central axis TRo. It is possible that the shape of the weight sequence be, for example, the Hamming window or a Hanning window.

In contrast, in a case in which the depth of the representative point Q is less than the transmission focal depth, it is possible to improve the S/N ratio of a signal. The reason is as follows. The user of the ultrasound diagnostic apparatus expects the S/N ratio of an ultrasound image (B-mode image) to be higher as the depth decreases. Therefore, the user tends to immediately misunderstand that the quality of an ultrasound image is low as the S/N ratio is low, regardless of whether the spatial resolution is high or low. For this reason, the weight sequence is set such that the ratio of a weight for the oscillator located at the center of the transmitting/receiving aperture TRx in the array direction to a weight for the oscillator located at the end of the transmitting/receiving aperture TRx is low, that is, the shape of the weight sequence is flat. It is possible that each weight in the weight sequence has a shape which does not depend on the distance from the transmitting/receiving aperture central axis TRo. The shape of the weight sequence may be, for example, a rectangular window.

The weight calculator 1047 calculates a weight sequence for each receiving oscillator Rm, using the information indicating the position of the transmitting/receiving aperture TRx which has been output from the target region setter 1042 as an input, and outputs the calculated weight sequence for each representative point Qk (hereinafter, a set of weight sequences for each depth is referred to as a “weighting profile”) to the adder 1048.

vii) Adder 1048

The adder 1048 receives, as inputs, the received signals identified in correspondence with each receiving oscillator Rm which have been output from the delay processor 1046 and the weighting profile output from the weight calculator 1047, multiplies the received signals identified in correspondence with each receiving oscillator Rm by the weights for each receiving oscillator Rm, and adds the received signals to generate an acoustic line signal for the representative point Qk. The delay processor 1046 may adjust the phase of the received signals detected by each receiving oscillator Rm in the transmitting/receiving aperture TRx and the adder 1048 may perform an addition process to superimpose the received signals received by each receiving oscillator Rm on the basis of the waves reflected from the representative point Qk, thereby increasing the S/N ratio of the signal. It is possible to extract the received signal from the representative point Qk.

The above-mentioned process is summarized as follows. It is assumed that a received signal sequence for the receiving oscillator Rm is Rf(m, t). Here, “m” is an identifier indicating the receiving oscillator and “t” is the time when the receiving oscillator Rc receives the ultrasonic waves reflected from the representative point Qk. In addition, it is assumed that a weighting coefficient for the receiving oscillator Rm is A(m). Furthermore, it is assumed that the difference between the reception time of the receiving oscillator Rm and the reception time of the receiving oscillator Rc is d(m). In this case, an acoustic line signal Das(k) for the representative point Qk is represented by the following expression.

$$Das ( k ) = {\sum }_{m}^{ } \{ A ( m ) \times Rf ( m , t + d ( m ) ) \} \qquad [ Expression 1 ]$$

The above-mentioned process can generate an acoustic line signal for all of the representative points Qk on the target line BL related to one transmission event. Then, an acoustic line signal is generated for all of the observation points Pij in the target region Bx on the basis of the acoustic line signal for the representative points Qk (which will be described in detail below). Then, the ultrasonic waves are repeatedly transmitted while the transmitting aperture Tx is sequentially moved in the array direction in synchronization with the transmission event and the transmission of the ultrasonic waves from all of the oscillators 101a in the probe 101 is performed to generate a frame acoustic line signal which is one frame of synthesized acoustic line signals.

In addition, hereinafter, a synthesized acoustic line signal for each observation point forming the frame acoustic line signal is referred to as a “synthesized acoustic line signal”.

The adder 1048 generates a line-region acoustic line signal for all of the representative points Qk on the target line BL in synchronization with the transmission event. The generated line-region acoustic line signal is output and stored in the data storage 107.

(3) Weighting Synthesizer 1140

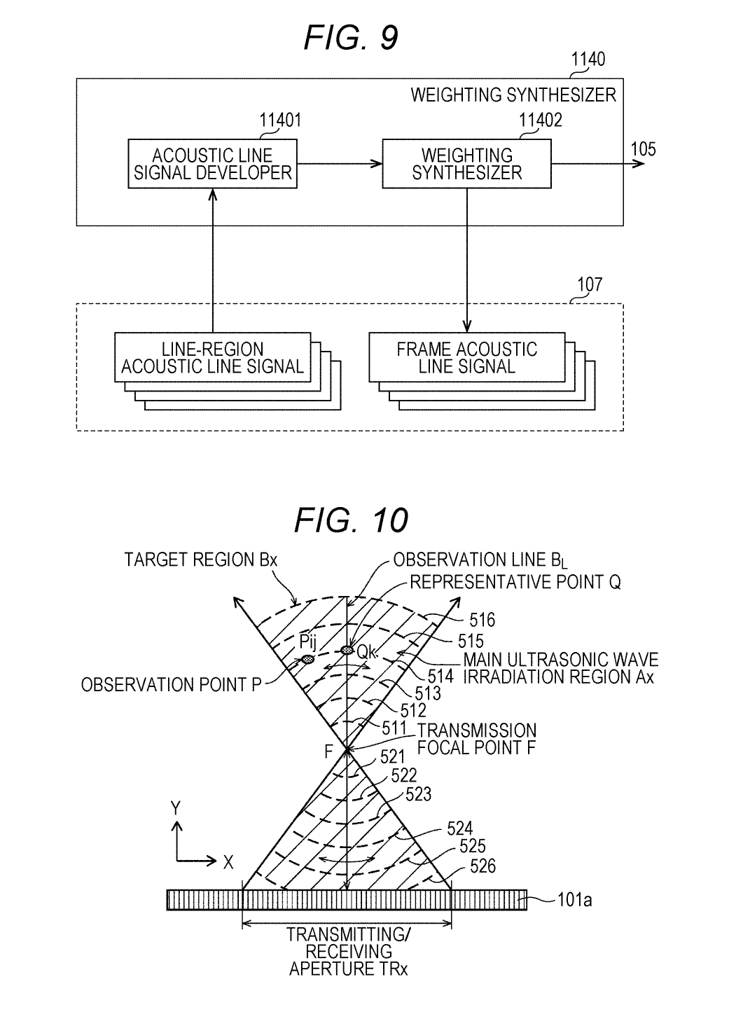

The weighting synthesizer 1140 is a circuit that generates a sub-frame acoustic line signal from the line-region acoustic line signal generated in synchronization with the transmission event and synthesizes the generated sub-frame acoustic line signals into the frame acoustic line signal. FIG. 9 is a functional block diagram illustrating the configuration of the weighting synthesizer 1140. As illustrated in FIG. 9, the weighting synthesizer 1140 includes an acoustic line signal developer 11401 and a weighting synthesizer 11402.

Next, the configuration of each component forming the weighting synthesizer 1140 will be described below.

i) Acoustic Line Signal Developer 11401

The acoustic line signal developer 11401 is a circuit that generates the sub-frame acoustic line signal and reads a plurality of line-region acoustic line signals stored in the data storage 107 after the generation of a series of line-region acoustic line signals for generating the frame acoustic line signal ends. Then, for each of the line-region acoustic line signals, the sub-frame acoustic line signals are generated from the line-region acoustic line signals on the basis of the positional relationship between the observation point Pij and the representative point Qk.

FIG. 10 is a diagram schematically illustrating a sub-frame acoustic line signal generation process of the acoustic line signal developer 11401. First, a case in which both the depth of the representative point Qk and the depth of the observation point Pij are greater than the transmission focal depth is assumed. As described above, the transmission time of the representative point Qk depends on the sum of the distance from the transmitting/receiving aperture TRx to the transmission focal point F and the distance from the transmission focal point F to the representative point Qk. That is, in a case in which the distance from the transmission focal point F to the representative point Qk is equal to the distance from the transmission focal point F to the observation point Pij, the transmission time of the representative point Qk is equal to the transmission time of the observation point Pij. Similarly, the reception time of the representative point Qk depends on the sum of the distance from the observation point Qk to the transmission focal point F and the distance from the transmission focal point F to the receiving oscillator Rm. That is, in a case in which the distance from the transmission focal point F to the representative point Qk is equal to the distance from the transmission focal point F to the observation point Pij, the reception time of the representative point Qk is equal to the reception time of the observation point Pij. Therefore, an acoustic line signal for the representative point Qk includes acoustic line signals for a plurality of observation points Pij which have the same distance to the transmission focal point F. In other words, the sum of the acoustic line signals for the plurality of observation points Pij having the same distance to the transmission focal point F is acquired as the acoustic line signal for the representative point Qk. This relationship is also established in a case in which both the depth of the representative point Qk and the depth of the observation point Pij are less than the transmission focal depth.

Therefore, the acoustic line signal developer 11401 applies the acoustic line signal for the representative point Qk as the value of the acoustic line signal for the observation point Pij which satisfies the following two conditions: (1) both the depth of the representative point Qk and the depth of the observation point Pij are greater or less than the transmission focal depth; and (2) the distance between the representative point Qk and the transmission focal point F is equal to the distance between the observation point Pij and the transmission focal point F. Specifically, an arc that has the transmission focal point F as the center and passes through the representative point Qk is set in the target region Bx and the value of the acoustic line signal for the representative point Qk on the art is applied as the values of the acoustic line signals corresponding to all of the observation points Pij on the arc. For example, the value of the acoustic line signal for the representative point Qk on an arc 514 is applied as the values of the acoustic line signals corresponding to all of the observation points Pij on the arc 514. The acoustic line signal developer 11401 generates the sub-frame acoustic line signal from the line-region acoustic line signals using this process.

The acoustic line signal developer 11401 outputs the generated sub-frame acoustic line signal to the weighting synthesizer 11402.

ii) Weighting Synthesizer 11402

The weighting synthesizer 11402 is a circuit that weights and synthesizes the sub-frame acoustic line signals into a frame acoustic line signal. FIG. 11 is a diagram schematically illustrating a synthesized acoustic line signal synthesis process of the weighting synthesizer 11402. As described above, the ultrasonic waves are sequentially transmitted and received while the oscillators used in a transmitting oscillator array (transmitting/receiving aperture TRx) are moved in the direction of the oscillator array by the pitch Mp in synchronization with the transmission event. Therefore, the position of the target region Bx based on a different transmission event is moved in the same direction by the pitch Mp for each transmission event. A plurality of sub-frame acoustic line signals are added, using the position of the observation points Pij corresponding to the acoustic line signals included in each sub-frame acoustic line signal as an index. In this way, the frame acoustic line signals included in the entire target region Bx are synthesized.

In this case, the weighting synthesizer 11402 weights the sub-frame acoustic line signal, using the position of the observation point Pij as an index. The weight sequence is a sequence of weigh coefficients applied to each sub-frame acoustic line signal corresponding to the observation point Pij. The weight sequence is defined by the position of the transmission focal point F in the transmission event corresponding to the sub-frame acoustic line signal. The weight sequence has a symmetric distribution with respect to the observation point Pij. The weight sequence is set such that the weight for the sub-frame acoustic line signal in the transmission event in which the transmission focal point F is set at the same X-coordinate (a position in the direction in which the oscillators are arranged) as that of the observation point Pij is the maximum. The central axis of the weight distribution is aligned with a straight line Pijo that passes through the observation point Pij and is perpendicular to the oscillator array. It is possible that the shape of the weight sequence is, for example, the Hamming window or the Hanning window. In this embodiment, a weight sequence 511 of which the distribution shape is the Hamming window, regardless of the depth of the observation point Pij, is used as the weight sequence. That is, the weighting profile includes only one weight sequence. The shape of the weight sequence may be, for example, a rectangular window without a weight distribution.

The weighting synthesizer 11402 weights the sub-frame acoustic line signals corresponding to each observation point Pij and adds the sub-frame acoustic line signals to generate a frame acoustic line signal.

In addition, for the observation points Pij in a plurality of target regions Bx at different positions, the values of the acoustic line signals in each sub-frame acoustic line signal are added. Therefore, the synthesized acoustic line signal has a large value according to the degree of overlap. Hereinafter, the number of times the observation point Pij is included in a different target region Bx is referred to as a “superimposition number” and the maximum value of the superimposition number in the direction of the oscillator array is referred to as a “maximum superimposition number”.

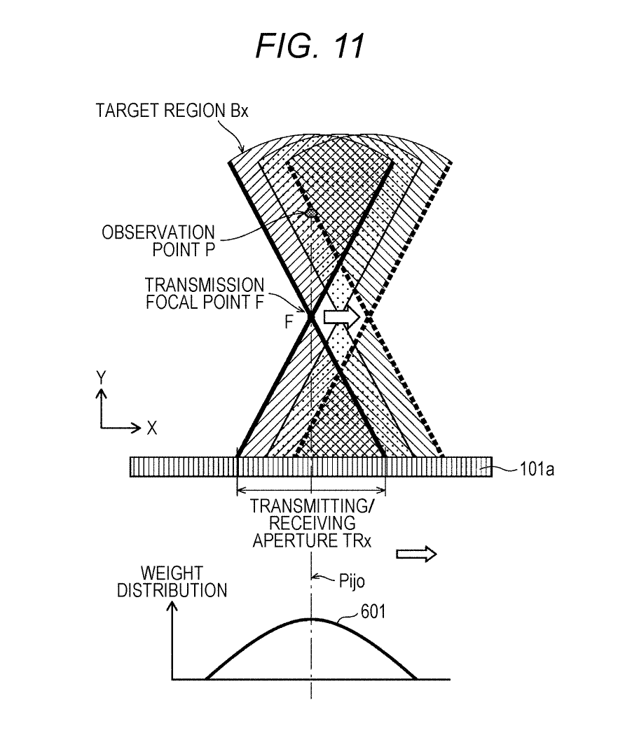

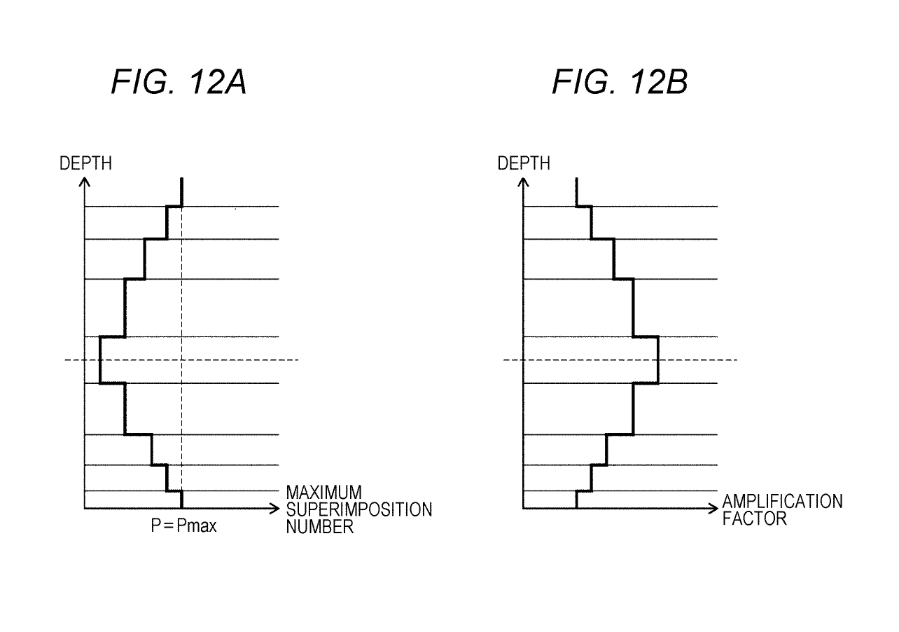

In this embodiment, the target region Bx is in a sandglass-shaped region. Therefore, as illustrated in FIG. 12A, since the superimposition number and the maximum superimposition number vary in the depth direction of the subject, the value of the synthesized acoustic line signal also varies in the depth direction. In order to compensate for the change, the weighting synthesizer 11402 performs an amplification process of multiplying each synthesized acoustic line signal by an amplification factor determined on the basis of the number of addition processes in the synthesis of the synthesized acoustic line signals included in the frame acoustic line signal.

FIG. 12B is a diagram schematically illustrating the amplification process of the weighting synthesizer 11402. As illustrated in FIG. 12B, the maximum superimposition number varies in the depth direction of the subject. Therefore, in order to compensate for the change, the synthesized acoustic line signal is multiplied by the amplification factor that varies in the depth direction of the subject depending on the maximum superimposition number. In this way, the cause of variation in the synthesized acoustic line signal associated with the change in the superimposition number in the depth direction is removed and the value of the synthesized acoustic line signal after the amplification process is uniformized in the depth direction.

In addition, a process may be performed which multiplies the synthesized acoustic line signal by an amplification factor that varies in the direction of the oscillator array depending on the superimposition number. In a case in which the superimposition number varies in the direction of the oscillator array, the cause of the variation is removed and the value of the synthesized acoustic line signal after the amplification process is uniformized in the direction of the oscillator array.

Furthermore, a signal obtained by performing the amplification process for the generated synthesized acoustic lines for each observation point may be used as the frame acoustic line signal.

<Operation>

The operation of the ultrasound diagnostic apparatus 100 having the above-mentioned configuration will be described below.

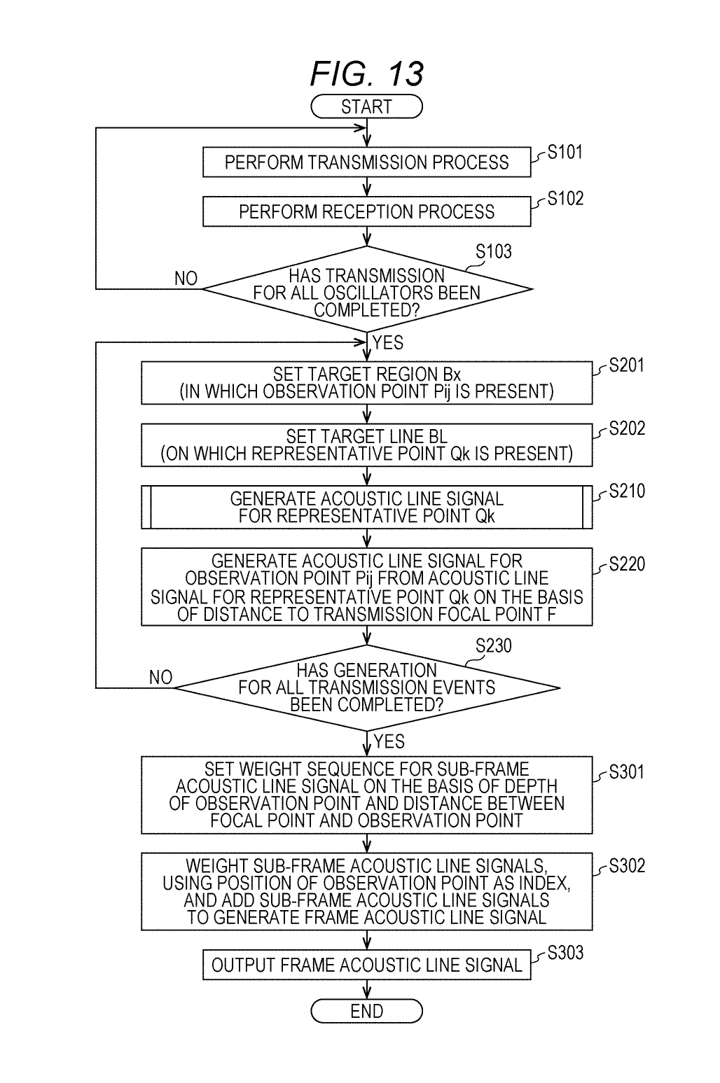

FIG. 13 is a flowchart illustrating the operation of a beam forming process of the reception beam former 104.

First, in step S101, the transmitter 1031 performs a transmission process (transmission event) that supplies a transmission signal for directing each oscillator included in the transmitting aperture Tx among the plurality of oscillators 101a in the probe 101 to transmit an ultrasound beam.

Then, in step S102, the receiver 1040 generates a received signal on the basis of an electric signal obtained from the reflected ultrasonic wave received by the probe 101, outputs the received signal to the data storage 107, and stores the received signal in the data storage 107. It is determined whether the transmission of the ultrasonic waves from all of the oscillators 101a in the probe 101 has been completed (step S103). In a case in which the transmission has not been completed, the process returns to step S101 and the transmission event is performed while the transmitting/receiving aperture TRx is being moved in the array direction by the pitch Mp. In a case in which the transmission has been completed, the process proceeds to step S201.

Then, in step S201, the target region setter 1042 sets the target region Bx on the basis of information indicating the position of the transmitting/receiving aperture TRx in synchronization with the transmission event. In a first loop, the target region Bx calculated from the transmitting/receiving aperture TRx in the first transmission event is set.

Then, in step S202, the target region setter 1042 sets the target line BL in the set target region Bx. The target line BL is a linear region that is present in the target region Bx and passes through the transmission focal point F.

Then, in step S210, an acoustic line signal is generated for the representative point Qk.

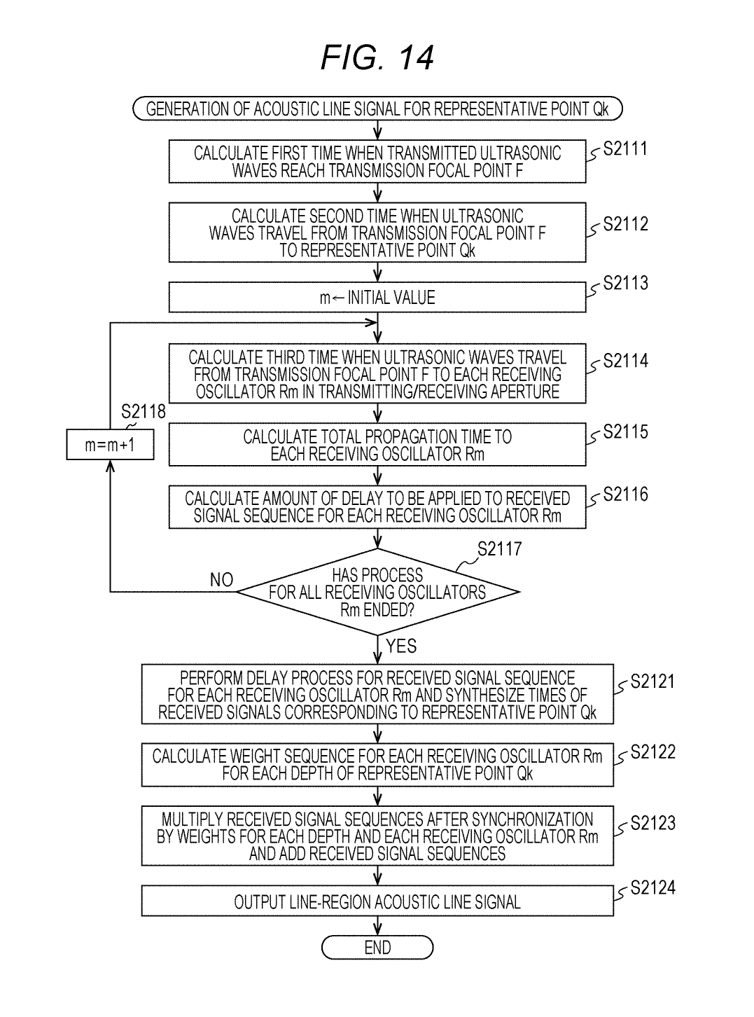

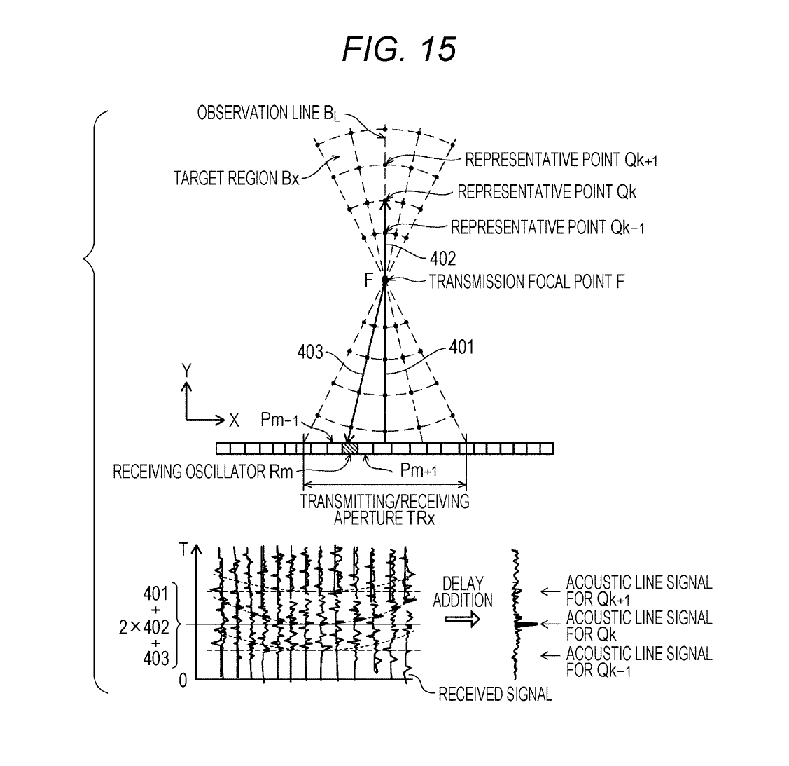

Here, the operation of generating the acoustic line signal for the representative point Qk in step S210 will be described below. FIG. 14 is a flowchart illustrating the operation of generating the acoustic line signal for the representative point Qk in the reception beam former 104. FIG. 15 is a diagram schematically illustrating the operation of generating the acoustic line signal for the representative point Qk in the reception beam former 104.

First, in step S2111, the transmission time calculator 1043 calculates a first time when the transmitted ultrasonic waves reach the transmission focal point F. The first time is calculated by dividing the length of the path (401) from the transmitting/receiving aperture TRx to the transmission focal point F which is geometrically determined by the velocity cs of the ultrasonic waves.

Then, in step S2112, the transmission time calculator 1043 calculates a second time when the ultrasonic waves travel from the transmission focal point F to the representative point Qk. The second time can be calculate by dividing the length of the path (402 or 405) from the transmission focal point F to the representative point Qk which is geometrically determined by the velocity cs of the ultrasonic waves. In a case in which the depth of the representative point Qk is less than the transmission focal depth, a negative value of which the absolute value is calculated is used as the second time. That is, for two representative points Qx and Qy having the same distance from the transmission focal point, it is assumed that the second time of the representative point Qx of which the depth is greater than the transmission focal depth is 1.5 μs and the second time of the representative point Qy of which the depth is less than the transmission focal depth is −1.5 μs.

The transmission time calculator 1043 calculates the sum of the first time and the second time as the transmission time for the representative point Qk, outputs the transmission time to the delay amount calculator 1045, and outputs the second time to the reception time calculator 1044.

Then, a coordinate m indicating the position of the receiving oscillator Rm which is calculated from the transmitting/receiving aperture TRx is initialized to the minimum value in the transmitting/receiving aperture TRx (step S2113) and the reception time when the transmitted ultrasonic waves are reflected from the representative point Qk in the subject and reach the receiving oscillator Rm in the transmitting/receiving aperture TRx is calculated. Here, the time when the ultrasonic waves reflected from the representative point Qk reach the transmission focal point F has already been calculated as the second time in step S2112. Therefore, the reception time calculator 1044 calculates a third time when the reflected ultrasonic waves travel from the transmission focal point F to the receiving oscillator Rm in the transmitting/receiving aperture TRx (step S2114). The third time can be calculated by dividing the length of the path 403 from the transmission focal point F to the receiving oscillator Rm which is geometrically determined by the velocity cs of the ultrasonic waves. Then, the reception time calculator 1044 outputs the sum of the second time and the third time as the reception time to the delay amount calculator 1045. The delay amount calculator 1045 calculates the total propagation time when the ultrasonic waves transmitted from the transmitting/receiving aperture TRx are reflected from the representative point Qk and reach the receiving oscillator Rm from the sum of the transmission time and the reception time (step S2115) and calculates the amount of delay for each receiving oscillator Rm, using a difference in the total propagation time between the receiving oscillators Rm in the transmitting/receiving aperture TRx (step S2116).

It is determined whether the calculation of the amounts of delay for all of the receiving oscillators Rm in the transmitting/receiving aperture TRx has been completed (step S2117). In a case in which the calculation has not been completed, the coordinate m is incremented (step S2118) and the calculation of the amount of delay for the receiving oscillator Rm is performed again (step S2114). In a case in which the calculation has been completed, the process proceeds to step S2121. In this stage, the amount of delay of the ultrasonic waves reflected from the representative point Qk is calculated for all of the receiving oscillators Rm in the transmitting/receiving aperture TRx.

In step S2121, the delay processor 1046 performs a delay process based on the amounts of delay for each receiving oscillator Rm for the received signal sequence corresponding to the receiving oscillators Rm in the transmitting/receiving aperture TRx and synchronizes the time (timing) of the received signals based on the waves reflected from the representative point Qk. As described above, in the total propagation time, the first time is uniquely determined by the positional relationship between the transmission focal point F and the transmitting/receiving aperture TRx, the second time is uniquely determined by the positional relationship between the transmission focal point F and the representative point Qk, and the third time is uniquely determined by the positional relationship between the transmission focal point F and the receiving oscillator Rm. Here, in one transmission event, both the position of the transmission focal point F and the position of the transmitting/receiving aperture TRx are fixed. Therefore, the first time is constant for all of the representative points Qk and all of the receiving oscillators Rm. In addition, the second time does not depend on the position of the receiving oscillator Rm. Therefore, for one receiving oscillator Rm, a difference in the total propagation time between the representative point Qk and a representative point Q(k+1) does not depend on the coordinate m. That is, in the received signal sequence corresponding to the same receiving oscillator, a difference in time between a received signal based on the waves reflected from the representative point Qk and a received signal based on the waves reflected from the representative point Q(k+1) depends on only the distance between the representative point Qk and the representative point Q(k+1) and there is no difference between a signal corresponding to the receiving oscillator Rm and a signal corresponding to a receiving oscillator R(m+1). In contrast, since the third time does not depend on the position of the representative point Qk, a difference in time between a received signal based on the waves reflected from the representative point Qk in a received signal sequence corresponding to the receiving oscillator Rm and a received signal based on the waves reflected from the representative point Qk in a received signal sequence corresponding to the receiving oscillator R(m+1) depends on only the positional relationship between the receiving oscillator Rm, the receiving oscillator R(m+1), and the transmission focal point F and there is no difference between the received signal based on the waves reflected from the representative point Qk and the received signal based on the waves reflected from the representative point Q(k+1). Therefore, when a delay process for removing the difference in the third time between the received signal sequences corresponding to each receiving oscillator Rm is performed, the times of the received signals based on the waves reflected from the representative point Qk are synchronized with each other, the times of the received signals based on the waves reflected from the representative point Q(k+1) are synchronized with each other, and the times of the received signals based on the waves reflected from the representative point Q(k−1) are synchronized with each other in the received signal sequences. Therefore, it is not necessary to identify the received signals on the basis of the total propagation time for each representative point Qk and the delay process based on the third time is performed for each received signal sequence to identify the received signals for each representative point Qk as the received signal sequence in the direction of the oscillator array on the basis of the first time and the second time.

Then, the weight calculator 1047 calculates a weight sequence for each receiving oscillator Rm (step S2122). As described above, the weight calculator 1047 applies the Hamming window in which the weight for the oscillator located at the center of the transmitting/receiving aperture TRx in the array direction is the maximum to the representative point Qk of which the depth is equal to or greater than the transmission focal depth and applies a rectangular window to the representative point Qk of which the depth is less than the transmission focal depth. The adder 1048 multiplies the received signal sequences identified in correspondence with each receiving oscillator Rm by the weight for each receiving oscillator Rm and adds the received signal sequences to generate an acoustic line signal for the representative point Qk (step S2123). The generated acoustic line signal for the representative point Qk is output and stored in the data storage 107 (step S2124).

Then, returning to FIG. 13, in step S220, the acoustic line signal developer 11401 generates an acoustic line signal for the observation point Pij (“⋅” in FIG. 15) in the target region Bx on the basis of the acoustic line signal for the representative point Qk. As described above, for the observation point Pij, the acoustic line signal developer 11401 specifies the representative point Qk of which the depth is greater than the transmission focal depth among the representative points Qk having the same distance from transmission focal point F in a case in which the depth of the observation point Pij is greater than the transmission focal depth and specifies the representative point Qk of which the depth is less than the transmission focal depth in a case in which the depth of the observation point Pij is less than the transmission focal depth. Then, the acoustic line signal developer 11401 uses the acoustic line signal for the specified representative point Qk as the acoustic line signal for the observation point Pij. In this stage, the sub-frame acoustic line signals for all of the observation points Pij in the target region Bx associated with one transmission event are generated.

Then, it is determined whether the generation of the sub-frame acoustic line signals for all of the transmission events has ended (step S230). In a case in which the generation has not ended, the process returns to step S201 and the target region Bx is set on the basis of the transmitting/receiving aperture TRx (step S201). In a case in which the generation has ended, the process proceeds to step S301.

Then, in step S301, the weighting synthesizer 11402 sets a weight sequence for the sub-frame acoustic line signals on the basis of the depth of the observation point Pij and the position of the transmission focal point F in a transmission event corresponding to the sub-frame acoustic line signal for the observation point Pij. As described above, the weighting synthesizer 11402 applies the Hamming window in which a weight for the sub-frame acoustic line signal corresponding to the transmission event in which the position of the transmission focal point F and the observation point Pij are on the same straight line perpendicular to the oscillator array is the maximum.

Then, in step S302, the weighting synthesizer 11402 weights a plurality of sub-frame acoustic line signals, using the position of the observation point Pij as an index, on the basis of the weight sequence, adds the sub-frame acoustic line signals to generate synthesized acoustic line signals for each observation point Pij, and synthesizes the synthesized acoustic line signals into a frame acoustic line signal. Then, the weighting synthesizer 11402 outputs the frame acoustic line signal to the ultrasound image generator 105 and the data storage 107 (step S303) and ends the process.

<Summary>

As described above, according to the ultrasound diagnostic apparatus 100 of this embodiment, the acoustic line signals for the observation point P at the same position which have been generated by different transmission events are superimposed and synthesized by the synthetic aperture method. Therefore, the effect of virtually performing transmission focus is also obtained for the observation point P with a depth different from the depth of the transmission focal point F in a plurality of transmission events. As a result, it is possible to improve spatial resolution and the S/N ratio of a signal.

In addition, in the ultrasound diagnostic apparatus 100, when the sub-frame acoustic line signal is generated, phasing addition is not performed for all of the observation points Pij, the representative point Qk is provided in each arc-shaped region having the transmission focal point F as the center, and phasing addition is performed for only the representative points Qk. Therefore, the number of representative points Qk to be subjected to phasing addition does not depend on the area of the target region Bx, but depends on the length of the target region Bx in the depth direction. As a result, it is possible to significantly reduce the amount of computation in phasing addition. In addition, since both the transmission time and the reception time are based on the transmission focal point F, it is not necessary to repeatedly perform the reception time calculation process for each receiving oscillator Rm for each representative point Qk. Therefore, it is possible to simplify both the total propagation time calculation process and the phasing addition process. As a result, it is possible to significantly reduce the amount of computation in phasing addition. In addition, it is possible to obtain the effect of improving spatial resolution and the S/N ratio of a signal by synthesizing different sub-frame acoustic line signals for the same observation point. Therefore, it is possible to prevent a reduction in spatial resolution and the S/N ratio of a signal according to the degree of reduction in the amount of computation.

Furthermore, in the ultrasound diagnostic apparatus 100, the weighting profile in which the weight sequence to be applied varies depending on whether the depth of the representative point Qk is greater or less than the transmission focal depth is used. In the weighting profile, in the region of which the depth is less than the transmission focal depth, the shape of the weight sequence is a flat shape in which a coefficient is little changed or is hardly changed, such as a rectangular window. In this way, in the region of which the depth is less than the transmission focal depth, the effect of improving the S/N ratio of a signal in the synthesis of the received signal sequences is sufficiently obtained. Therefore, in the region of which the depth is less than the transmission focal depth and in which the user expects the high S/N ratio of the signal, it is possible to prevent a reduction in the S/N ratio of the signal and to avoid the underestimation of the quality of an ultrasound image which is contrary to the expectations of the user. In contrast, in the region of which the depth is greater than the transmission focal depth, the weight sequence has a shape in which the weight for the center is the maximum, that is, the shape of the weight sequence is, for example, the Hamming window. Therefore, a large weight sequence is applied to the oscillator that can receive the waves reflected from the observation point Pij with the highest sensitivity, that is, the oscillator closest to the transmission focal point F. As a result, it is possible to achieve a high spatial resolution in the region of which the depth is greater than the transmission focal depth.

### Modification Example 1

In the ultrasound diagnostic apparatus 100 according to Embodiment 1, the weight calculator 1047 uses the weighting profile in which a Hamming-window-shaped weight sequence is used for the representative point Qk of which the depth is equal to or greater than the transmission focal depth and a rectangular-window-shaped weight sequence is used for the representative point Qk of which the depth is less than the transmission focal depth. However, the weighting profile may satisfy the following conditions and is not limited to the example described in Embodiment 1.

As the weighting method during phasing addition, the following methods are considered: (A) a method in which the weight increases as the distance to the transmission focal point F decreases; and (B) a method that multiplies all of the oscillators by the same weight. According to the method (A), when there is a difference between a received signal obtained by an oscillator close to the transmission focal point F and a received signal obtained by an oscillator far away from the transmission focal point F, the result close to the received signal obtained by the oscillator that is close to the transmission focal point F is obtained. The acoustic line signal is mainly formed by the received signals that are less affected by the degradation of propagation in the subject. Therefore, it is possible to reduce the influence of the degradation of propagation in the subject and to improve the spatial resolution of the acoustic line signal. The received signals corresponding to the oscillators at both ends of the transmitting/receiving aperture TRx or the oscillators close to both ends do not contribute to the acoustic line signal. Therefore, in particular, in a case in which the number of receiving oscillators in the transmitting/receiving aperture TRx is small, the effect of improving the S/N ratio by removing noise components is likely to be insufficient. In contrast, according to the method (B), since the received signals corresponding to all of the oscillators contribute to the acoustic line signal, it is possible to maximize the effect of improving the S/N ratio by removing noise components. On the other hand, since weighting is not performed for the received signal that is less affected by the degradation of propagation and the received signal that is greatly affected by the degradation of propagation in the subject, in some cases, the spatial resolution of the acoustic line signal is not sufficiently improved due to the influence of the propagation-degraded received signal.

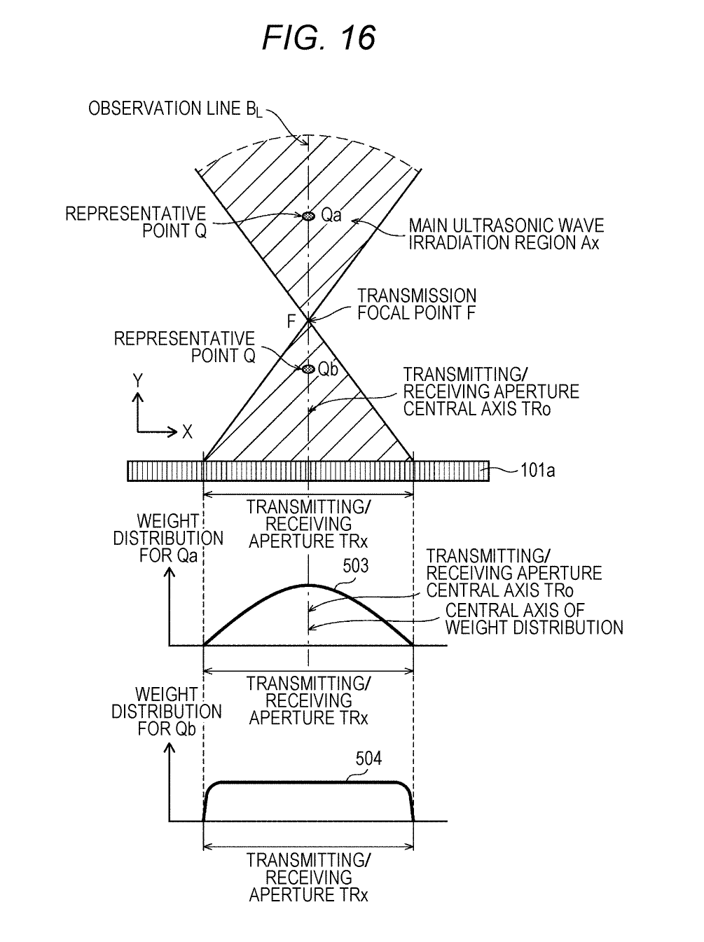

Therefore, for a weight sequence Wd for a representative point Q1 of which the depth is equal to or greater than the transmission focal depth and a weight sequence Ws for a representative point Q2 of which the depth is less than the transmission focal depth, when a weighting profile in which the weight sequence Wd has the tendency of (A) rather than the weight sequence Ws and the weight sequence Ws has the tendency of (B) rather than the weight sequence Wd is used, it is possible to obtain the same effect as that in Embodiment 1. That is, a weight for the oscillator located in the periphery may be smaller than a weight for the oscillator located at the center in the weight sequence Wd and the weight sequence Wd may tend to have a larger weight turbulence than the weight sequence Ws. For example, as illustrated in FIG. 16, a weight sequence for the representative point Q1 of which the depth is equal to or greater than the transmission focal depth may be a Hamming window 503 and a weight sequence for the representative point Q2 of which the depth is less than the transmission focal depth may be a weight sequence 504. Alternatively, the weight sequence for the representative point Q1 of which the depth is equal to or greater than the transmission focal depth may be a weight sequence 504 and the weight sequence for the representative point Q2 of which the depth is less than the transmission focal depth may be a rectangular window 502.

In Embodiment 1 and Modification Example 1, the same weight sequence as that for the representative point Q of which the depth is equal to or greater than the transmission focal depth is used for the representative point Qk of which the depth is equal to the transmission focal depth. However, the same weight sequence as that for the representative point Q of which the depth is less than the transmission focal depth may be used for the representative point Qk of which the depth is equal to the transmission focal depth. That is, the weight sequence may change depending on whether the depth of the representative point is greater or less than the transmission focal depth.

### Embodiment 2

In Embodiment 1 and Modification Example 1, the weight calculator 1047 uses the weighting profile in which a weight increases as the distance to the transmission focal point F decreases for the representative point Qk of which the depth is equal to or greater than the transmission focal depth rather than for the representative point Qk of which the depth is less than the transmission focal depth and the weighting synthesizer 11402 multiplies the same weight, regardless of the depth of the observation point Pij. However, the process of changing the weight sequence according to the depth does not need to be performed when the sub-frame acoustic line signal is generated and may be performed when the sub-frame acoustic line signals are synthesized.

In an ultrasound diagnostic apparatus according to Embodiment 2, a weight calculator multiplies the same weight, regardless of the depth of the representative point Qk and a weighting synthesizer uses a weighting profile in which a weight for the sub-frame acoustic line signal increases as the transmission focal point F is closer to the observation point Pij for the observation point Pij of which the depth is equal to or greater than the transmission focal depth rather than for the observation point Pij of which the depth is less than the transmission focal depth.

In the ultrasound diagnostic apparatus according to Embodiment 2, the weight calculator multiplies the same weight, regardless of the depth of the representative point Qk. Therefore, unlike Embodiment 1, the weight calculator uses a predetermined weight sequence, regardless of whether the depth of the representative point Qk is greater or less than the transmission focal depth. The predetermined weight sequence may have any shape in which a weight for the oscillator located at the center of the transmitting/receiving aperture TRx is the maximum, such as the Hamming window or the Hanning window. Alternatively, the predetermined weight sequence may be a rectangular window.

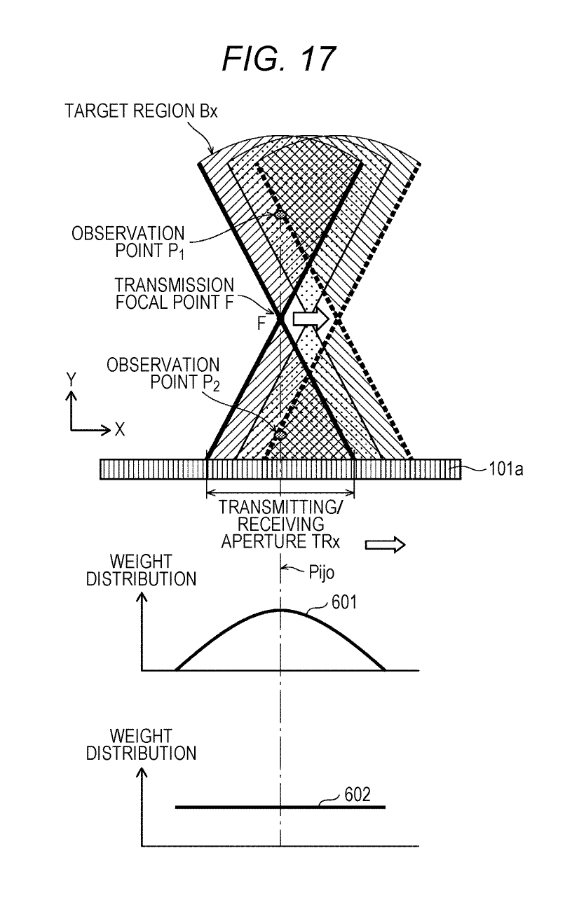

The weighting synthesizer uses a weight sequence in which a weight for the sub-frame acoustic line signal increases as the transmission focal point F is closer to the observation point Pij for the observation point Pij of which the depth is equal to or greater than the transmission focal depth rather than for the observation point Pij of which the depth is less than the transmission focal depth. Specifically, as illustrated in the schematic diagram of FIG. 17, a weight sequence 601 of which the distribution shape is the Hamming window is used for an observation point P1 of which the depth is equal to or greater than the transmission focal depth. In contrast, a weight sequence 602 of which the distribution shape is a rectangular window is used for an observation point P2 of which the depth is less than the transmission focal depth.

The weighting profile is not limited to the above-mentioned example. In a case in which the depth of the observation point Pij is equal to or greater than the transmission focal depth, it is possible that the weight is reduced depending on the distance between the observation point Pij and the transmission focal point F in the transmission event corresponding to the sub-frame acoustic line signal. It is possible that the shape of the weight sequence be, for example, the Hamming window or the Hanning window. In a case in which the depth of the observation point Pij is less than the transmission focal depth, it is possible that the shape of the weight sequence be a flat shape which does not depend on the distance between the observation point Pij and the transmission focal point F in the transmission event corresponding to the sub-frame acoustic line signal. It is possible that the shape of the weight sequence be, for example, a rectangular window. Similarly to Modification Example 1, in a case in which the shape of the weight sequence when the depth of the observation point Pij is less than the transmission focal depth is not a rectangular window, the weight sequence for the observation point P1 of which the depth is equal to or greater than the transmission focal depth may be a Hamming window 603 and the weight sequence for the observation point P2 of which the depth is less than the transmission focal depth may be a weight sequence 604, as illustrated in FIG. 18.

<Summary>

As described above, according to the ultrasound diagnostic apparatus of this embodiment, similarly to Embodiment 1, the effect of virtually performing transmission focus is also obtained for the observation point P with a depth different from the depth of the transmission focal point F in a plurality of transmission events. As a result, it is possible to improve spatial resolution and the S/N ratio of a signal. In addition, it is possible to significantly reduce the amount of computation in phasing addition and to prevent a reduction in the spatial resolution and the S/N ratio of the signal according to the degree of reduction in the amount of computation.

In the ultrasound diagnostic apparatus according to this embodiment, the weight sequence for the sub-frame acoustic line signal is changed depending on whether the depth of the observation point Pij is equal to or greater than the transmission focal depth or is less than the transmission focal depth. In the region of which the depth is less than the transmission focal depth, the shape of the weight sequence is a flat shape in which a coefficient is little changed or is hardly changed, such as a rectangular window. In this way, the effect of improving the S/N ratio of a signal in the synthesis of the sub-frame acoustic line signals is improved. Therefore, in the region of which the depth is less than the transmission focal depth and in which the user expects the S/N ratio of the signal to be high, it is possible to prevent a reduction in the S/N ratio of the signal and to avoid the underestimation of the quality of an ultrasound image which is contrary to the expectations of the user. In contrast, in the region of which the depth is greater than the transmission focal depth, the weight sequence has a shape in which the weight for the center is the maximum, that is, the shape of the weight sequence is, for example, the Hamming window. Therefore, a large weight sequence is applied to the sub-frame acoustic line signals in a situation in which the largest number of waves are reflected from the observation point Pij, that is, in the transmission event in which the observation point Pij is closest to the transmission focal point F. As a result, it is possible to achieve a high spatial resolution.

<<Effect of the Present Disclosure>>

Next, the effect of the embodiment will be described with reference to the comparison between the quality of an ultrasound image in reception beam forming according to the embodiment and the quality of an ultrasound image in reception beam forming according to a comparative example.

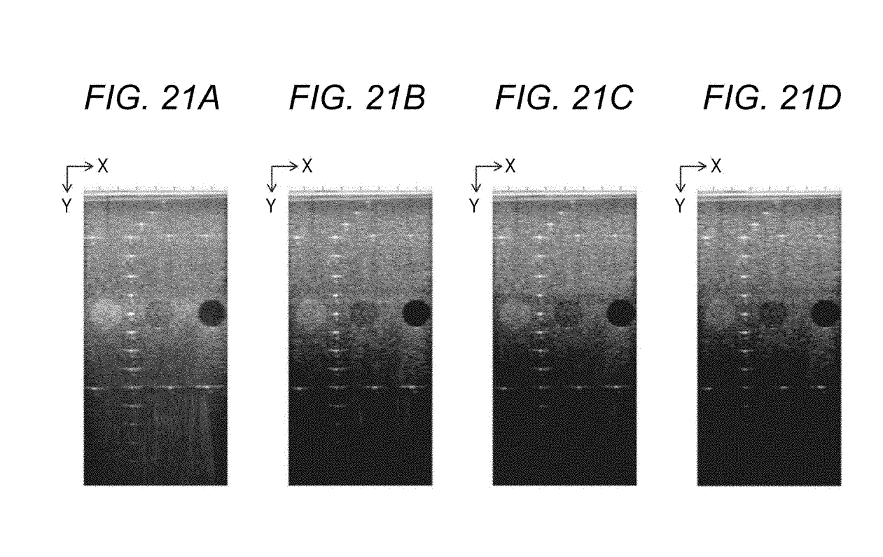

FIGS. 21A to 21D illustrate the comparison between ultrasound images in the reception beam forming according to comparative examples. FIGS. 21A to 21D illustrate the ultrasound images (B-mode tomographic images) of the same pseudo subject (phantom) captured by reception beam forming according to Comparative Examples 1 to 4, respectively. An ultrasound beam travels from the upper side to the lower side of the drawings.

In Comparative Examples 1 to 4, the reception beam forming process is the same as that in Embodiments 1 and 2 and each modification example except the weight sequence used by the weight calculator and the weight sequence used by the weighting synthesizer. In each comparative example, only one type of weight sequence used by the weight calculator and only one type of weight sequence used by the weighting synthesizer are used, regardless of the depth of the representative point Qk and the depth of the observation point Pij. Specifically, the weight sequence used by the weight calculator during phasing addition is a rectangular window in Comparative Examples 1 and 2 and is the Hamming window in Comparative Examples 3 and 4. In contrast, the weight sequence used by the weighting synthesizer during the synthesis of the sub-frame acoustic line signals is a rectangular window in Comparative Examples 1 and 3 and is the Hamming window in Comparative Examples 2 and 4.

As described above, in some cases, when strong weighting in which a weight at the center is greater than that in the periphery is performed using the weight sequence with a large turbulence, the spatial resolution is improved, but the S/N ratio is not sufficiently improved. In contrast, in some cases, when a flat weight sequence is used or when weighting is not performed, the S/N ratio is significantly improved, the spatial resolution is not sufficiently improved. Therefore, as illustrated in FIGS. 21A to 21D, the spatial resolution illustrated in FIG. 21A is the highest, followed by the spatial resolution illustrated in FIG. 21B, the spatial resolution illustrated in FIG. 21C, and the spatial resolution illustrated in FIG. 21D. The S/N ratio illustrated in FIG. 21D is the highest, followed by the S/N ratio illustrated in FIG. 21C, the S/N ratio illustrated in FIG. 21B, and the S/N ratio illustrated in FIG. 21A. Therefore, in Comparative Example 4 in which strong weighting is not performed, in the region of which the depth is less than the transmission focal depth, the S/N ratio is not sufficient and the spectrum is rough as illustrated in FIG. 21D. As a result, bleeding that is stronger than that illustrated in FIGS. 21A to 21C occurs in the X direction (the direction of an element array). In general, the user expects a higher spatial resolution and a higher S/N ratio in a shallower region. In addition, the user feels that the accuracy of other items, such as spatial resolution, is low in an image with a low S/N ratio. Therefore, when seeing the image illustrated in FIG. 21D in which the S/N ratio is low in a shallow region, the user falsely recognizes that the spatial resolution is low and underestimates the quality of the image. In contrast, in Comparative Example 1 in which weighting is not performed, the S/N ratio is high and the spatial resolution is low in the entire region. In particular, bleeding occurs in the X direction (the direction of the element array) in a region deeper than the focal point. Therefore, for the quality of the entire image, it is possible that a small weight be given to the region of which the depth is less than the transmission focal depth and a large weight be given to the region of which the depth is greater than the transmission focal depth.

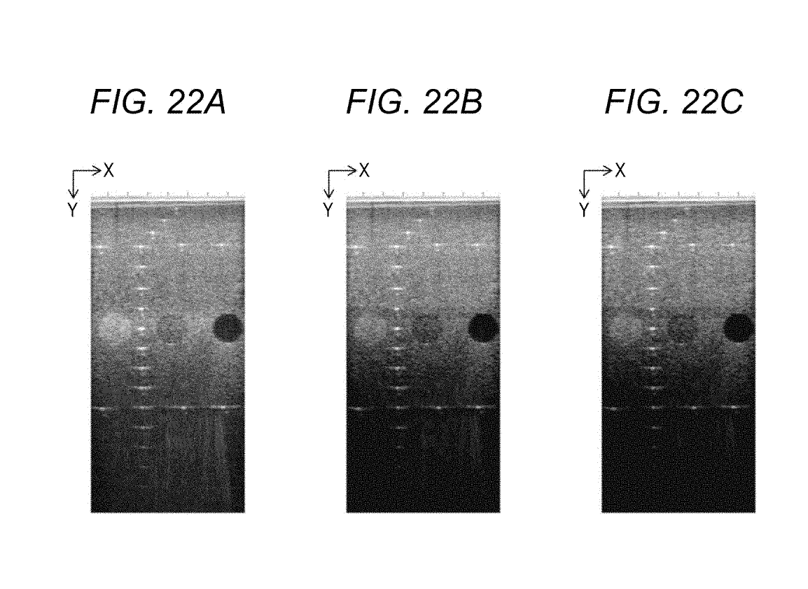

FIGS. 22A to 22C illustrate the comparison between Embodiment 1 and Comparative Examples 1 and 3. FIGS. 22A, 22B, and 22C correspond to Comparative Example 1, Embodiment 1, and Comparative Example 3, respectively. However, in FIG. 22B, the weight sequence used by the weighting synthesizer is set to a rectangular window as in Comparative Example 1 and Comparative Example 3. In Embodiment 1, as illustrated in FIG. 22B, a high S/N ratio in Comparative Example 1 can be selectively extracted in a shallow region and a high spatial resolution in Comparative Example 3 can be selectively extracted in a deep region. It is possible to achieve a better balance between the S/N ratio and the spatial resolution than that in the comparative examples.

### Embodiment 3

In Embodiment 1, the window shape of the weight sequence used by the weight calculator during phasing addition is changed depending on whether the depth of the representative point is less or greater than the transmission focal depth. In Embodiment 2, the window shape of the weight sequence used by the weighting synthesizer during the synthesis of the sub-frame acoustic line signals is changed depending on whether the depth of the representative point is less or greater than the transmission focal depth. In contrast, in this embodiment, a case in which the window shape of the weight sequence is changed only in the vicinity of the transmission focal depth will be described.

An ultrasound diagnostic apparatus according to Embodiment 3 is characterized in that the weight calculator and/or the weighting synthesizer uses a weight sequence with a different shape for the representative point Qk or the observation point Pij of which the depth is equal to the transmission focal depth.

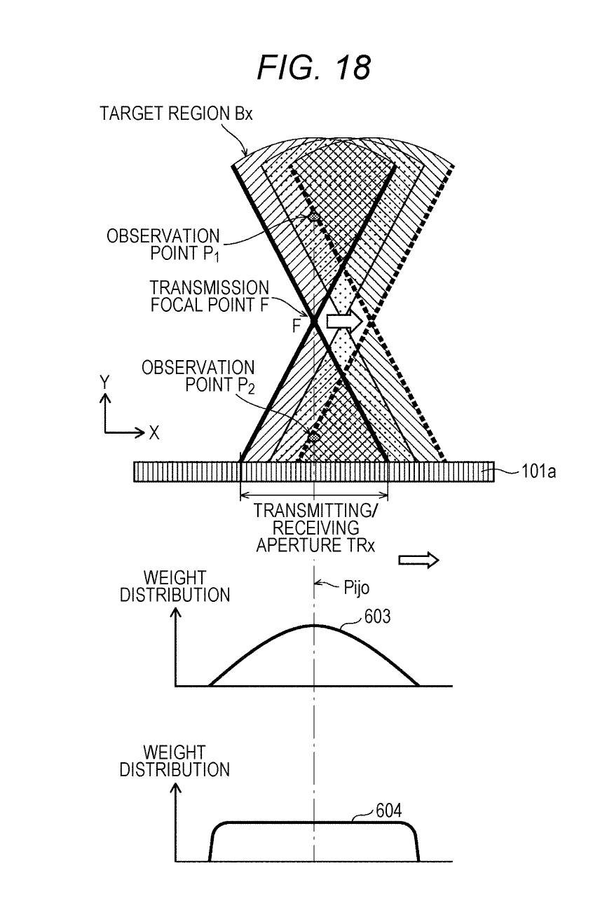

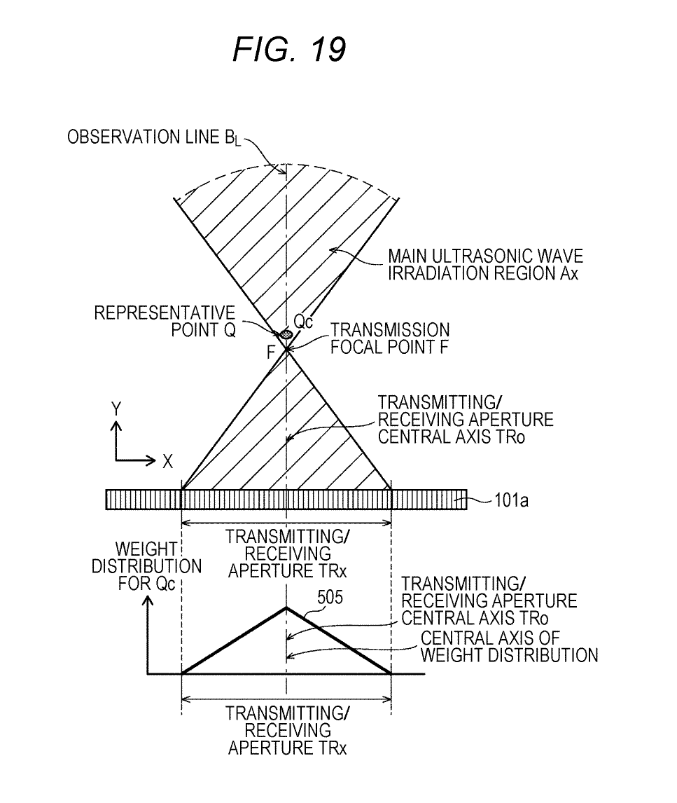

A case in which the weight calculator changes the weight sequence will be described with reference to FIG. 19. As illustrated in FIG. 19, the weight calculator applies a triangular window (Bartlett window) 505 as the weight sequence to a representative point Qc of which the depth is equal to the transmission focal depth. Similarly to the Hamming window or the Hanning window, in the triangular window, a weight for the oscillator at the center is greater than a weight for the oscillator at the end and a difference between the weight for the oscillator at the center and the weight for the oscillator in the vicinity of the center is more than that in the Hamming window or the Hanning window. Therefore, the weight for the oscillator at the center of the transmitting/receiving aperture RTx is further increased by the use of the triangular window.

The depth of the representative point Qc equal to the transmission focal depth means that representative point Qc and the observation point Pij sharing the acoustic line signal are located in the vicinity of the transmission focal point F. Therefore, a transmitted ultrasound beam is focused in the vicinity of the representative point Qc. When there is a reflection source of the ultrasonic waves at the representative point Qc, strong reflected waves are reflected. In addition, the oscillator located at the center of the transmitting/receiving aperture TRx is closest to the representative point Qc. Therefore, it is assumed that the quality of the received signal obtained for the representative point Qc by the oscillator located at the center of the transmitting/receiving aperture TRx is very high. A large weight is given to maximize the spatial resolution. In this way, it is possible to improve signal quality in the vicinity of the transmission focal depth.

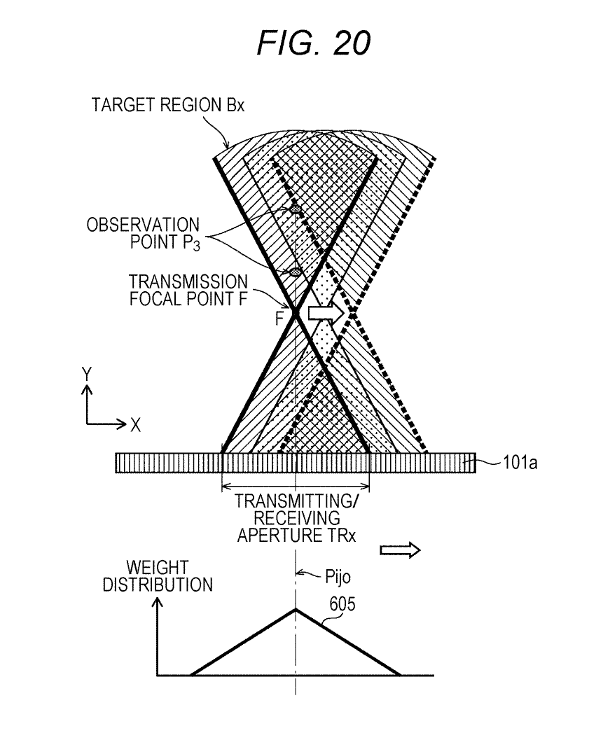

As illustrated in FIG. 20, the weighting synthesizer may apply the triangular window (Bartlett window) 605 for the observation point Pij of which the depth is equal to the transmission focal depth. In this case, a weight for a sub-frame acoustic line signal in a transmission event in which the observation point Pij is closest to the transmission focal point F is maximized Therefore, it is possible to maximize a weight for a sub-frame acoustic line signal with the highest quality and to improve signal quality in the vicinity of the transmission focal depth.

### Other Modification Examples of Embodiments

(1) In each embodiment and each modification example, the transmission time calculator 1043 calculates the first time when the ultrasonic waves transmitted from the transmitting/receiving aperture TRx reach the transmission focal point F and the second time when the ultrasonic waves travel from the transmission focal point F to the representative point Qk, the reception time calculator 1044 adds the second time and the third time when the ultrasonic waves travel from the transmission focal point F to the receiving oscillator Rm, and the delay amount calculator 1045 adds the transmission time and the reception time. However, for example, the transmission time calculator may calculate the first time and the second time and output the first time and the second time to the delay amount calculator. The reception time calculator may perform only the process of calculating the third time and output the third time to the delay amount calculator. The delay amount calculator may calculate the total propagation time on the basis of the first time, the second time, and the third time.

(2) In each embodiment and each modification example, the phasing adder 1041 generates the line-region acoustic line signals for each transmission event and stores the line-region acoustic line signals in the data storage 107 and the weighting synthesizer 1140 converts the line-region acoustic line signals for each transmission event into the sub-frame acoustic line signals for each transmission event and synthesizes the sub-frame acoustic line signals. However, for example, the acoustic line signal developer may store the generated sub-frame acoustic line signals in the data storage and the weighting synthesizer may read the sub-frame acoustic line signals from the data storage. Alternatively, for example, the acoustic line signal developer may not be provided in the weighting synthesizer, but may be provided in the phasing adder. The phasing adder may store the sub-frame acoustic line signals for each transmission event in the data storage and the weighting synthesizer may read the sub-frame acoustic line signals from the data storage. With this configuration, it is easy to process phasing addition, the generation of the sub-frame acoustic line signal, and the synthesis of the acoustic line signals in parallel, using different, and it is easy to achieve a configuration for improving a calculation speed.

In addition, the phasing adder 1041 starts to generate the line-region acoustic line signal after a plurality of transmission events corresponding to one frame end. However, for example, the phasing adder 1041 may generate the line-region acoustic line signal whenever a receiving process related to the transmission event is completed. In addition, the acoustic line signal developer may generate the sub-frame acoustic line signal immediately after the line-region acoustic line signal is generated. With this configuration, when phasing addition and the generation of the sub-frame acoustic line signal are performed in parallel using different processors, it is possible to reduce the response time from the start of the transmission of ultrasonic waves to the generation of an ultrasound image.

(3) In Embodiment 1, the weight calculator 1047 creates the weighting profile in which the weight sequence changes before and after the focal depth. In Embodiment 2, the weighting synthesizer creates the weighting profile. In Embodiment 1, the weighting synthesizer 11402 uses a single weight sequence. In Embodiment 2, the weight calculator uses a single weight sequence. However, both the weight calculator and the weighting synthesizer may create the weighting profile in which the weight sequence changes before and after the focal depth. With this configuration, it is possible to optimize the balance between the spatial resolution and the S/N ratio in each of the region of which the depth is less than the focal depth and the region of which the depth is greater than the focal depth. In addition, in a case in which one of the weight calculator and the weighting synthesizer uses a single weight sequence, the coefficient of the weight sequence may use any window function other than the Hamming window, the Hanning window, and the rectangular window in order to change the balance between the spatial resolution and the S/N ratio to a desired state.

(4) In each embodiment and each modification example, the transmitting aperture set by the transmitter 1031 and the receiving aperture set by the receiver 1040 are completely matched with each other as the transmitting/receiving aperture TRx. However, the receiving aperture is not necessarily matched with the transmitting aperture. For example, the receiving aperture may be a portion of the transmitting aperture. On the contrary, the width of the receiving aperture may be greater than the width of the transmitting aperture. However, it is possible that the central axis of the transmitting aperture be aligned with the central axis of the receiving aperture.

(5) The invention has been described on the basis of various embodiments. However, the invention is not limited to the embodiments and also includes the following cases.

For example, one or more embodiments of the invention may be applied to a computer system including a microprocessor and a memory. The memory may store the computer program and the microprocessor may operate on the basis of the computer program. For example, one or more embodiments of the invention may be applied to a computer system that includes a computer program for the ultrasound signal processing method according to one or more embodiments of the invention and operates according to the program (or instructs each unit connected thereto to perform operations).

One or more embodiments of the invention also include a case in which a portion of or the entire the ultrasound diagnostic apparatus and a portion of or the entire ultrasound signal processing device are implemented by a computer system including a microprocessor, a recording medium, such as a ROM or a RAM, and a hard disk unit. The RAM or the hard disk unit stores a computer program for implementing the same operations as those of each of the above-mentioned devices. The microprocessor operates on the basis of the computer program to implement the functions of each device.

Some or all of the components forming each of the devices may be implemented by one system large scale integration (LSI). The system LSI is a multi-function LSI manufactured by integrating a plurality of components into one chip. Specifically, the system LSI is a computer system including, for example, a microprocessor, a ROM, and a RAM. These components may be individually integrated into one chip or some or all of the components may be integrated into one chip. In addition, the LSI is referred to as an IC, a system LSI, a super LSI, or an ultra LSI according to the degree of integration. The RAM stores a computer program for implementing the same operations of as those of each of the above-mentioned devices. The microprocessor operates on the basis of the computer program to implement the functions of the system LSI. For example, one or more embodiments of the invention also include a case in which the beam forming method according to the invention is stored as a program of an LSI and the LSI is inserted into a computer and executes a predetermined program (beam forming method).

A method for manufacturing an integrated circuit is not limited to the LSI. The method may be implemented by a dedicated circuit or a general-purpose processor. After the LSI is manufactured, a field programmable gate array (FPGA) or a reconfigurable processor in which the connection or settings of circuit cells of an LSI are reconfigurable may be used.

Furthermore, when an integrated circuit technique that replaces the LSI is developed with the progress of semiconductor technology or by other techniques derived from the semiconductor technology, the technique may be used to integrate functional blocks.

In addition, a processor, such as a CPU, may execute a program to implement some or all of the functions of the ultrasound diagnostic apparatus according to each embodiment. The invention may be applied to a non-transitory computer-readable recording medium that stores a program for implementing a diagnostic method for the ultrasound diagnostic apparatus or a beam forming method. A program or a signal may be recorded on a recording medium and the recording medium may be provided such that the program is executed by another independent computer system. In addition, the program may be distributed through a transmission medium such as the Internet.

In the ultrasound diagnostic apparatus according to the above-described embodiment, the data storage which is a storage device is provided in the ultrasound diagnostic apparatus. However, the storage device is not limited thereto. For example, a semiconductor memory, a hard disk drive, an optical disk drive, or a magnetic storage device may be connected to the ultrasound diagnostic apparatus from the outside.

The division of the functional blocks in the block diagram is an illustrative example. A plurality of functional blocks may be integrated into one functional block, one functional block may be divided into a plurality of functional blocks, or some functions may be transferred to other functional blocks. In addition, the functions of a plurality of functional blocks with similar functions may be processed in parallel or in a time-division manner by a single hardware component or software.

The order of the above-mentioned steps is an illustrative example for describing the invention in detail. The steps may be performed in other orders. In addition, some of the steps may be performed at the same time as (in parallel to) other steps.

In the ultrasound diagnostic apparatus, the probe and the display are connected from the outside. However, the probe and the display may be integrally provided in the ultrasound diagnostic apparatus.

In the above-described embodiments, the probe has a probe configuration in which a plurality of piezoelectric elements are one-dimensionally arranged. However, the configuration of the probe is not limited thereto. For example, a two-dimensional array probe in which a plurality of piezoelectric elements are two-dimensionally arranged or a swing-type probe in which a plurality of oscillators that are one-dimensionally arranged are mechanically swung to acquire a three-dimensional tomographic image may be used. The probe may be appropriately used according to measurement methods. For example, in a case in which the two-dimensional array probe is used, the time when a voltage is applied to the piezoelectric element or the value of the voltage is changed to control the irradiation position or direction of the ultrasound beam to be transmitted.

Furthermore, the probe may have some of the functions of a transceiver. For example, the following configuration can be used. A transmission electric signal is generated in the probe on the basis of a control signal for generating the transmission electric signal output from the transceiver and the transmission electric signal is converted into ultrasonic waves. In addition, the received reflected ultrasonic waves are converted into a received electric signal and a received signal is generated on the received electric signal in the probe.

Furthermore, at least some of the functions of the ultrasound diagnostic apparatuses according to each embodiment and the modification examples may be combined with each other. All of the above-mentioned numerical numbers are an illustrative example for describing the invention in detail. The invention is not limited to the exemplified numerical numbers.

In addition, it will be understood by those skilled in the art that various modifications of the embodiments can be made without departing from the scope and spirit of the invention and the invention also may include the modifications.

### SUMMARY

(1) An ultrasound signal processing device according to an embodiment repeatedly performs a transmission event, which transmits a focused ultrasound beam to a subject using an ultrasound probe including a plurality of oscillators, a plurality of times, receives reflected ultrasonic waves from the subject in synchronization with each transmission event, generates a received signal sequence, and synthesizes a plurality of sub-frame acoustic line signals generated on the basis of the received reflected ultrasonic waves to obtain an acoustic line signal. The ultrasound signal processing device includes: a transmitter that selects a transmitting/receiving oscillator array from the plurality of oscillators arranged in a line in the ultrasound probe and directs the transmitting/receiving oscillator array to transmit the ultrasound beam to a target region of the subject for each transmission event, while changing a focal point defining a focus position of the ultrasound beam for each transmission event; a receiver that generates a received signal sequence for each oscillator included in the transmitting/receiving oscillator array on the basis of the reflected ultrasonic waves received from the target region by the ultrasound probe in synchronization with each transmission event; a phasing adder that generates a line-region acoustic line signal from the received signal sequence, using a weighting, phasing, and addition process including a delay process based on a distance between the focal point and each of a plurality of observation points on a straight line passing through the focal point and a distance between the focal point and the oscillator and a weighting process based on a first weighting profile, for each transmission event; an acoustic line signal developer that allocates the line-region acoustic line signal of an observation point, which has the same distance from the focal point as each observation point in the target region and is on the straight line, as the acoustic line signal of the observation point to generate the sub-frame acoustic line signal for each transmission event; and a weighting synthesizer that weights a plurality of sub-frame acoustic line signals related to a plurality of transmission events on the basis of the position of the observation point, using a second weighting profile, and synthesizes the plurality of sub-frame acoustic line signals into a frame acoustic line signal. In at least one of the first weighting profile and the second weighting profile, a first weight sequence for a first observation point deeper than the focal point has a larger turbulence than a second weight sequence for a second observation point shallower than the focal point.

An ultrasound signal processing method according to another embodiment repeatedly performs a transmission event, which transmits a focused ultrasound beam to a subject using an ultrasound probe including a plurality of oscillators, a plurality of times, receives reflected ultrasonic waves from the subject in synchronization with each transmission event, generates a received signal sequence, and synthesizes a plurality of sub-frame acoustic line signals generated on the basis of the received reflected ultrasonic waves to obtain an acoustic line signal. The ultrasound signal processing method includes: selecting a transmitting/receiving oscillator array from the plurality of oscillators arranged in a line in the ultrasound probe and directing the transmitting/receiving oscillator array to transmit the ultrasound beam to a target region of the subject for each transmission event, while changing a focal point defining a focus position of the ultrasound beam for each transmission event; generating a received signal sequence for each oscillator included in the transmitting/receiving oscillator array on the basis of the reflected ultrasonic waves received from the target region by the ultrasound probe in synchronization with each transmission event; generating a line-region acoustic line signal from the received signal sequence, using a weighting, phasing, and addition process including a delay process based on a distance between the focal point and each of a plurality of observation points on a straight line passing through the focal point and a distance between the focal point and the oscillator and a weighting process based on a first weighting profile, for each transmission event; allocating the line-region acoustic line signal of an observation point, which has the same distance from the focal point as each observation point in the target region and is on the straight line, as the acoustic line signal of the observation point to generate the sub-frame acoustic line signal for each transmission event; and weighting a plurality of sub-frame acoustic line signals related to a plurality of transmission events on the basis of the position of the observation point, using a second weighting profile, and synthesizing the plurality of sub-frame acoustic line signals into a frame acoustic line signal. In at least one of the first weighting profile and the second weighting profile, a first weight sequence for a first observation point deeper than the focal point has a larger turbulence than a second weight sequence for a second observation point shallower than the focal point.

According to the configuration or the method, it is possible to obtain the effect of improving the spatial resolution and the S/N ratio of a signal by virtually performing transmission focus using the synthetic aperture method and it is possible to significantly reduce the amount of computation by performing phasing addition only for a representative point which is one of the observation points. In addition, phasing addition and weighting in the development of an acoustic line signal are appropriately controlled by the configuration according to the embodiment of the invention to adjust the balance between the spatial resolution and the S/N ratio of a signal. As a result, it is possible to obtain a high-quality ultrasound tomographic image.

(2) In the ultrasound signal processing device according to (1), a ratio of a weighting coefficient at an end in an element array direction to a weighting coefficient at a center in the element array direction in the second weight sequence may be less than a ratio of a weighting coefficient at an end in the element array direction to a weighting coefficient at a center in the element array direction in the first weight sequence.

With this configuration, it is possible to mainly obtain the effect of improving the S/N ratio of a signal in a region shallower than the focal point and to mainly obtain the effect of improving spatial resolution in a region deeper than the focal point.

(3) In the ultrasound signal processing device according to (2), in the first weight sequence, the weighting coefficient may become smaller as it becomes further away from the center in the element array direction.

(4) In the ultrasound signal processing device according to (3), the first weight sequence may be a Hamming window.

With these configurations, it is possible to improve spatial resolution in the region deeper than the focal point.

(5) In the ultrasound signal processing device according to any one of (2) to (4), the second weight sequence may be a rectangular window in which a value of the weighting coefficient does not change in the element array direction.

(6) In the ultrasound signal processing device according to (5), the first weighting profile may be a Hamming window for the first observation point deeper than the focal point and may be a rectangular window for the second observation point shallower than the focal point. The second weighting profile may be a rectangular window.

With these configurations, it is possible to maximize the effect of improving the S/N ratio of a signal in the region shallower than the focal point and to prevent the user from falsely recognizing that the quality of an ultrasound image is degraded.

(7) In the ultrasound signal processing device according to any one of (1) to (6), in at least one of the first weighting profile and the second weighting profile, a third weight sequence in which a weighting coefficient is reduced depending on a distance from a center in the element array direction may be used for a third observation point in the vicinity of the focal point.

(8) In the ultrasound signal processing device according to (7), the third weight sequence may be a triangular window in which the weighting coefficient has a maximum value at the center and is 0 at an end in the element array direction.

With these configurations, it is possible to maximize the effect of improving spatial resolution in a region with the same depth as the focal point.

(9) In the ultrasound signal processing device according to any one of (1) to (8), in a case in which the depth of the observation point is equal to or greater than a focal depth where the ultrasonic waves are focused in the subject, the phasing adder may calculate, as a transmission time when the transmitted ultrasonic waves reach each observation point, a sum of a first time when the ultrasonic waves transmitted from the transmitting oscillator array reach the focal point and a second time when the transmitted ultrasonic waves travel from the focal point to the observation point. In a case in which the depth of the observation point is less than the focal depth where the ultrasonic waves are focused in the subject, the phasing adder may calculate, as the transmission time, a result obtained by subtracting the second time from the first time.

With this configuration, it is possible to calculate the delay time for each observation point with higher accuracy.

(10) In the ultrasound signal processing device according to any one of (1) to (9), the phasing adder may calculate a time when the ultrasonic waves reflected from the observation point reach an oscillator closest to the observation point as a reception time corresponding to the oscillator closest to the observation point and may add a difference between a time when the ultrasonic waves travel from the focal point to an oscillator and a time when the ultrasonic waves travel from the focal point to the oscillator closest to the observation point to the reception time corresponding to the oscillator closest to the observation point to calculate the reception time corresponding to the oscillator.

With this configuration, it is possible to more simply calculate the delay time for each observation point and to reduce the amount of computation. In addition, the combination between (10) and (9) makes it possible to simplify a phasing addition operation and thus to reduce the amount of computation.

The ultrasound signal processing device, the ultrasound diagnostic apparatus, the ultrasound signal processing method, the program, and the non-transitory computer-readable recording medium according to the present disclosure are useful to improve the performance of the ultrasound diagnostic apparatus according to the related art, particularly, to reduce the cost or computational load of an arithmetic device, thereby improving a frame rate. In addition, the present disclosure may be applied to, for example, sensors using a plurality of array elements, in addition to the ultrasonic waves.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. An ultrasound signal processing device that repeatedly performs a transmission event, which transmits a focused ultrasound beam to a subject using an ultrasound probe including a plurality of oscillators, a plurality of times, receives reflected ultrasonic waves from the subject in synchronization with each transmission event, generates a received signal sequence, and synthesizes a plurality of sub-frame acoustic line signals generated on the basis of the received reflected ultrasonic waves to obtain an acoustic line signal, the ultrasound signal processing device comprising:
a transmitter that selects a transmitting/receiving oscillator array from the plurality of oscillators arranged in a line in the ultrasound probe and directs the transmitting/receiving oscillator array to transmit the ultrasound beam to a target region of the subject for each transmission event, while changing a focal point defining a focus position of the ultrasound beam for each transmission event;
a receiver that generates a received signal sequence for each oscillator included in the transmitting/receiving oscillator array on a basis of the reflected ultrasonic waves received from the target region by the ultrasound probe in synchronization with each transmission event;
a phasing adder that generates a line-region acoustic line signal from the received signal sequence, using a weighting, phasing, and addition process including a delay process based on a distance between the focal point and each of a plurality of observation points on a straight line passing through the focal point and a distance between the focal point and the oscillator and a weighting process based on a first weighting profile, for each transmission event;
an acoustic line signal developer that allocates the line-region acoustic line signal of an observation point, which has the same distance from the focal point as each observation point in the target region and is on the straight line, as the acoustic line signal of the observation point to generate the sub-frame acoustic line signal for each transmission event; and
a weighting synthesizer that weights the plurality of sub-frame acoustic line signals related to the plurality of transmission events on a basis of the position of the observation point, using a second weighting profile, and synthesizes the plurality of sub-frame acoustic line signals into a frame acoustic line signal,
wherein, in at least one of the first weighting profile and the second weighting profile, a first weight sequence for a first observation point deeper than the focal point has a larger turbulence than a second weight sequence for a second observation point shallower than the focal point.

2. The ultrasound signal processing device according to claim 1,
wherein a ratio of a weighting coefficient at an end in an element array direction to a weighting coefficient at a center in the element array direction in the second weight sequence is less than a ratio of a weighting coefficient at an end in the element array direction to a weighting coefficient at a center in the element array direction in the first weight sequence.

3. The ultrasound signal processing device according to claim 2,
wherein, in the first weight sequence, the weighting coefficient becomes smaller as it becomes further away from the center in the element array direction.

4. The ultrasound signal processing device according to claim 3,
wherein the first weight sequence is a Hamming window.

5. The ultrasound signal processing device according to claim 2,
wherein the second weight sequence is a rectangular window in which a value of the weighting coefficient does not change in the element array direction.

6. The ultrasound signal processing device according to claim 5,
wherein the first weighting profile is a Hamming window for the first observation point deeper than the focal point and is a rectangular window for the second observation point shallower than the focal point, and
the second weighting profile is a rectangular window.

7. The ultrasound signal processing device according to claim 1,
wherein, in at least one of the first weighting profile and the second weighting profile, a third weight sequence in which a weighting coefficient is reduced depending on a distance from a center in an element array direction is used for a third observation point in the vicinity of the focal point.

8. The ultrasound signal processing device according to claim 7,
wherein the third weight sequence is a triangular window in which the weighting coefficient has a maximum value at the center and is 0 at an end in the element array direction.

9. The ultrasound signal processing device according to claim 1,
wherein, in a case in which a depth of the observation point is equal to or greater than a focal depth where the ultrasonic waves are focused in the subject, the phasing adder calculates, as a transmission time when transmitted ultrasonic waves reach each observation point, a sum of a first time when the ultrasonic waves transmitted from the transmitting/receiving oscillator array reach the focal point and a second time when the transmitted ultrasonic waves travel from the focal point to the observation point, and
in a case in which the depth of the observation point is less than the focal depth where the ultrasonic waves are focused in the subject, the phasing adder calculates, as the transmission time, a result obtained by subtracting the second time from the first time.

10. The ultrasound signal processing device according to claim 1,
wherein, for a reception time when the ultrasonic waves reflected from each observation point reach each oscillator, the phasing adder calculates a time when the ultrasonic waves reflected from the observation point reach an oscillator closest to the observation point as the reception time corresponding to the oscillator closest to the observation point and adds a difference between a time when the ultrasonic waves travel from the focal point to an oscillator and a time when the ultrasonic waves travel from the focal point to the oscillator closest to the observation point to the reception time corresponding to the oscillator closest to the observation point to calculate the reception time corresponding to the oscillator.

11. An ultrasound diagnostic apparatus comprising:
an ultrasound probe; and
the ultrasound signal processing device according to claim 1.

12. An ultrasound signal processing method that repeatedly performs a transmission event, which transmits a focused ultrasound beam to a subject using an ultrasound probe including a plurality of oscillators, a plurality of times, receives reflected ultrasonic waves from the subject in synchronization with each transmission event, generates a received signal sequence, and synthesizes a plurality of sub-frame acoustic line signals generated on a basis of the received reflected ultrasonic waves to obtain an acoustic line signal, the method comprising:
selecting a transmitting/receiving oscillator array from the plurality of oscillators arranged in a line in the ultrasound probe and directing the transmitting/receiving oscillator array to transmit the ultrasound beam to a target region of the subject for each transmission event, while changing a focal point defining a focus position of the ultrasound beam for each transmission event;
generating the received signal sequence for each oscillator included in the transmitting/receiving oscillator array on a basis of the reflected ultrasonic waves received from the target region by the ultrasound probe in synchronization with each transmission event;
generating a line-region acoustic line signal from the received signal sequence, using a weighting, phasing, and addition process including a delay process based on a distance between the focal point and each of a plurality of observation points on a straight line passing through the focal point and a distance between the focal point and the oscillator and a weighting process based on a first weighting profile, for each transmission event;
allocating the line-region acoustic line signal of an observation point, which has the same distance from the focal point as each observation point in the target region and is on the straight line, as the acoustic line signal of the observation point to generate the sub-frame acoustic line signal for each transmission event; and
weighting the plurality of sub-frame acoustic line signals related to the plurality of transmission events on a basis of the position of the observation point, using a second weighting profile, and synthesizing the plurality of sub-frame acoustic line signals into a frame acoustic line signal,
wherein, in at least one of the first weighting profile and the second weighting profile, a first weight sequence for a first observation point deeper than the focal point has a larger turbulence than a second weight sequence for a second observation point shallower than the focal point.
